(19) United States
(12) Patent Application Publication
(10) Pub. No.: US 2024/0394873 A1
(43) Pub. Date: Nov. 28, 2024

(54) METHOD, SYSTEM AND COMPUTER-ACCESSIBLE MEDIUM EXTRACTING AND ANALYZING ELECTROGRAM FEATURES USING ARTIFICIAL INTELLIGENCE TO PREDICT SUCCESSFUL TREATMENT SITE(S)

(71) Applicant: THE TRUSTEES OF COLUMBIA UNIVERSITY IN THE CITY OF NEW YORK, New York, NY (US)

(72) Inventors: AMARDEEP SALUJA, Brooklyn, NY (US); CHRISTINE HENDON, Bronx, NY (US); ZIYI HUANG, New York, NY (US)

(21) Appl. No.: 18/665,782

(22) Filed: May 16, 2024

Related U.S. Application Data
(60) Provisional application No. 63/466,885, filed on May 16, 2023.

(51) Int. Cl. G06T 7/00; A61B 18/00; A61B 18/14

(52) U.S. Cl. CPC G06T 7/0012; A61B 2018/00577; A61B 2018/00839; A61B 18/1492; G06T 2207/20076; G06T 2207/20084; G06T 2207/30048

(57) ABSTRACT

Exemplary systems, methods, and computer-accessible medium are provided that can facilitate an electrogram feature set associated with an object. Thus, the exemplary systems, methods, and computer-accessible medium can be provided that can receive image information for the object and determine at least one characteristic of at least one portion of the object based on the image information and a time series voltage trace. The exemplary systems, methods, and computer-accessible medium are provided that can localize a reentry isthmus. Thus, exemplary systems, methods, and computer-accessible medium can be provided that can receive image information for an object and generate a probability map for the object based on a plurality of characteristics related to an electrogram feature set, and using the image information.

### CROSS REFERENCE TO RELATED APPLICATION(S)

This application relates to and claims the benefit of priority from U.S. Provisional Patent Application No. 63/466,885, filed on May 16, 2023, the entire disclosure of which is incorporated herein by reference in its entirety.

### STATEMENT REGARDING FEDERALLY FUNDED RESEARCH

This invention was made with government support under Grant No. HL127776, awarded by the National Institutes of Health. The government has certain rights in this invention.

### FIELD OF THE DISCLOSURE

The present disclosure relates to systems, methods and computer-accessible medium for (i) extracting and analyzing electrogram features using artificial intelligence to predict successful treatment site(s), and (ii) providing an automated prediction of isthmus areas in scar-related ventricular tachycardia (VT) using artificial intelligence.

### BACKGROUND INFORMATION

Cardiac arrhythmias are common and cause significant morbidity and mortality. Ablation is a commonly used modality to treat arrhythmias.

A common mechanism of arrhythmia generation involves circus-movement rotation of electricity called reentry. Such activity as produced when the electricity that causes cardiac contraction travels in a circuit. The critical component of such a circuit is typically an area of diseased tissue that is isolated form the rest of the myocardium by functional or anatomic barriers, such as regions of scarring or a valve annulus. One of the main goals of an ablation procedure can be to determine the location of such critical components (frequently termed “isthmuses”).

One of the tools that are currently used to characterize such circuits involves the use of electroanatomic mapping systems, which are commercial systems that keep track of the position of a recording catheter as it is moved around various positions in the heart and the electrical activation recorded at those positions. Such systems allow the operator to draw a map of electrical activation. The electroanatomic map, in conjunction with other information frequently obtained through pacing maneuvers, can provide the operator more specific information about the structure of the circuit which can aid the operator in selecting isthmus targets for ablation. (See, e.g., Ref. 1).

This process can be time consuming, and may not be possible to perform when the arrhythmia in question is dangerous to the patient or transient. Techniques have been developed to predict isthmus locations from information obtained when the patient is not in arrhythmia, (see, e.g., Ref. 2), but even with these techniques, the overall arrhythmia-free success rate of many ablations remains low.

Ablation of scar-related reentrant VT—which can be associated with high recurrence rates—is challenging, as the rhythm is hemodynamically unstable. Durable freedom from VT requires ablation of protected isthmus areas. Although substrate-based strategies have become common, VT ablation is still done in VT in some institutions. Recurrence is common and improved strategies to identify ablation targets are needed. Recently, machine learning (ML) has been more commonly used in medicine. However, the data collected during ablations is not spatially ordered in a manner utilized by common ML techniques. A graph convolutional network (GCN) is a type of ML model that can process spatially heterogenous data. GCN can be trained to identify isthmus areas in VT ablation. The GCN's input was a vector of features custom-calculated from the sampled electrograms (EGMs).

Structural and electrical properties of isthmuses in arrhythmia. Prior studies of arrhythmia in the ventricle (ventricular tachycardia or VT) after a heart attack have established that isthmus areas are composed of regions of viable tissue in otherwise damaged areas, identified by voltage measurement as scar. (See, e.g., Ref. 9). Electricity travels across the isthmus before exiting, typically in the border zone between normal myocardium and scar. In both animal models and humans, the signal has been seen to rotate around a region where electricity cannot conduct because of anatomic or functional electrical block before reentering the isthmus area (see FIG. 1). (See, e.g., Refs. 3 and 4). Human mapping studies of VT have established that when measured during arrhythmia, VT isthmus regions contain low-amplitude signals that are typically measured during segments of the electrocardiogram that is otherwise electrically silent (diastole). (See, e.g., Ref 5). High-density mapping studies have shown that conduction velocity is relatively preserved in isthmus zones, while regions preceding and following the isthmus have comparatively slower velocities (see, e.g., Ref. 6) which are typically accompanied by longer duration electrograms. (See, e.g., Ref. 7)

Electrical properties of isthmuses in normal rhythm. When assessed in during a normal rhythm, proto-isthmus regions have been noted to display signals that are shorter in duration in comparison to surrounding signals. (See, e.g., Refs. 8 and 9). Regions of slow electrical propagation, have been seen to correspond to critical isthmuses (see, e.g., Ref. 10), the ablation of which has been shown to be associated with freedom from clinical VT in humans. (See, e.g., Ref. 7).

Isthmuses in atrial tachycardias (ATs). Many of the concepts used to describe VTs can be applicable to arrhythmias that originate in the atrium as well. The presence of AT isthmuses in patients with atrial scarring was identified during the early history of arrhythmia ablation. (See, e.g., Ref. 12). More recently, isthmus locations and characteristics of ATs in patients with prior ablation for atrial fibrillation has been described. (See, e.g., Ref. 13). Current-day mapping systems provide a much higher density of sampled points. Such high density mapping studies have shown atrial isthmuses to be associated with lower voltage than non-isthmus regions. (See, e.g., Ref. 14). In contrast to VTs, conduction velocity in atrial isthmus areas has been shown to be decreased compared to entry or exit regions. (See, e.g., Ref. 20). Other electrogram features have proven useful, such as the presence of double-electrocardiograms, which have been shown to correspond to areas of conduction block, and would be expected to be adjacent to isthmus locations. (See, e.g., Ref. 15).

Further, there is a need to provide techniques that will facilitate electrophysiologists to more rapidly identify isthmus sites. The rapid identification of an important isthmus can facilitate untolerated or transient arrhythmia to be ablated more successfully than previously, reduce the time these frequently critically patients are kept under anesthesia, and improve ablation success rates for thousands of patients that suffer from this disease. Further, it may be beneficial to provide exemplary systems, methods and computer-accessible mediums, which can overcome at least the deficiency described herein above.

### SUMMARY OF EXEMPLARY EMBODIMENTS

Feature extraction by custom code: The available data suggests that isthmus locations can be identified by the characteristics of the electrical signals recorded in those areas (see FIG. 2). Commercial electroanatomic mapping systems export the data obtained during as a series of hundreds to tens thousands of individual files which contain electrical signal and location information for each point. In initial work, we have developed custom Python code capable of collating this data for each sampled point.

Exemplary embodiments: According to the exemplary embodiment of the present disclosure, it is possible to provide exemplary systems, methods and computer-accessible mediums for (i) extracting and analyzing electrogram features using artificial intelligence to predict successful treatment site(s), and (ii) providing an automated prediction of isthmus areas in scar-related ventricular tachycardia using artificial intelligence. In one exemplary embodiment of the present disclosure, a graph convolutional network (GCN) structure can be used to process electrogram (EM) point clouds for network training and isthmus prediction in VT cases. For example, such exemplary systems, methods and computer-accessible mediums can be used to assess and accuracy of a trained GCN in predicting isthmus regions in scar-related reentrant VT.

To that end, exemplary systems, methods, and non-transitory computer accessible medium according to certain exemplary embodiments of the present disclosure can be provided which can facilitate the development of an electrogram feature set associated with an object by receiving image information for the object and determining at least one characteristic of at least one portion of the object based on the image information and a time series voltage trace. In some embodiments, the determination of the at least one characteristic uses one or more distances between sampling points between adjacent sections of the object.

Furthermore, an exemplary embodiment may rely on a machine learning procedure to determine the at least one characteristic. In some embodiments, this characteristic or characteristics is related to a reentry isthmus location. Also, in some embodiments, the at least one characteristic is determined from an electrogram of a patient undergoing a catheter ablation procedure of an atrial tachycardia or ventricular tachycardia.

Exemplary systems, methods, and non-transitory computer accessible medium according to certain exemplary embodiments of the present disclosure can be provided which can facilitate the localizing of a reentry isthmus by receiving image information for an object and generating a probability map for the object based on a plurality of characteristics related to an electrogram feature set, and using the image information. In some embodiments, the probability map is generated using a graph-convolutional neural network (GCN), and the GCN may be trained on the plurality of characteristics.

Furthermore, in some exemplary embodiments, the GCN generates the probability map at least partially based on a plurality of irregularly spaced electrogram sampling points. In exemplary embodiments, the probability map is used to predict or generate a location and a shape of the reentry isthmus.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is an exemplary VT mapped during an ablation procedure according to an exemplary embodiment of the present disclosure;

FIG. 2 is a set of exemplary electrograms taken from different sections of a left ventricle during an atrial tachycardia ablation and the derivation of width, deflections, and double potential spacing features according to an exemplary embodiment of the present disclosure;

FIG. 3 is three representative points sampled from the VT shown in FIG. 1, whereas a top panel is the unprocessed recorded electrogram, the middle panel is a normalized electrogram representing one cardiac cycle, and the lower panel represents the single cardiac cycle electrogram processed with an instantaneous energy operator in order to detect individual electrogram deflections according to an exemplary embodiment of the present disclosure;

FIG. 4 is an example of two mapped atrial tachycardias shown with visual representations of their feature values according to an exemplary embodiment of the present disclosure;

FIG. 5 is a set of exemplary maps from a single patient mapped in both sinus rhythm (left) and VT (right) according to an exemplary embodiment of the present disclosure;

FIG. 6 is a set of exemplary bar charts illustrating that a combination of LAT range, total signal width, median conduction vector angle, and voltage width and vector coherence can help distinguish isthmus from non-isthmus areas and different parts of the isthmus from each other according to an exemplary embodiment of the present disclosure;

FIG. 7 is an exemplary set of exemplary bar charts and graphs illustrating areas under the receiver operating characteristic curve (AUC) for association with isthmus location among eight features measured in AT, VT, and VT mapped in the presenting non-VT rhythms according to an exemplary embodiment of the present disclosure;

FIG. 8 is a set of exemplary graphs illustrating that for ventricular tachycardia patients studied in sinus rhythm, the measurement of local activation time (LAT) interquartile range (IQR) and LAT median absolute deviation (MAD) in isthmus and non-isthmus points (top panels) and the receiver-operator curves (ROC) for these measurements in distinguishing isthmus from non-isthmus points according to an exemplary embodiment of the present disclosure;

FIGS. 9(a)-9(d) are illustrations of a set of exemplary quantitative visualization of ILAM maps (left) with 8 and 1000 isochrones of FIGS. 9(a) and 9(b) respectively, interquartile activation range of FIG. 9(c) and mean absolute deviation of activation timing of FIG. 9(d), with the middle panels showing the isthmus in red, and a set of corresponding exemplary graphs illustrating area under the curve for each metric according to an exemplary embodiment of the present disclosure;

FIGS. 10(A) and 10(B) are illustrations of exemplary flow diagram and structure of an exemplary graph convolutional network, respectively, according to an exemplary embodiment of the present disclosure;

FIGS. 11A-11D are exemplary maps illustrating Omnipolar-derived features shown on atrial maps (FIGS. 11A and 11B) and ventricular maps (FIGS. 11C and 11D)-maps illustrated in FIGS. 11A and 11C illustrate Omnipolar-derived activation direction vectors plotted on top of colored isochrones, and maps illustrated in FIGS. 11B and 11D illustrate Omnipolar-derived conduction velocity maps according to an exemplary embodiment of the present disclosure;

FIG. 12A is an exemplary graph illustrating the robustness of an exemplary model to introduced noise as measured by the ratio of the likelihood ratios (LR) when the noise is introduced compared to baseline LR with different levels if introduced noise according to an exemplary embodiment of the present disclosure;

FIG. 12B is an exemplary graph illustrating the accuracy of an exemplary model as measured by centroid-centroid distance as it relates to point density according to an exemplary embodiment of the present disclosure;

FIGS. 13A-13D are exemplary visualizations of features extracted from electroanatomic maps from AT according to an exemplary embodiment of the present disclosure;

FIGS. 14A-14D are exemplary illustrations or activation and isthmus probability maps derived from maps within AT where probability maps (right illustrations of each) are shown next to activation maps (left illustrations of each) according to an exemplary embodiment of the present disclosure;

FIG. 15 is an illustration of an exemplary grouping of high-probability isthmus points into areas and visualization of the primary and secondary outcomes according to an exemplary embodiment of the present disclosure;

FIG. 16 is a set of exemplary bar charts illustrating primary (Centroid-Centroid distance) and secondary outcomes (e.g., Edge-Edge distance and Dice coefficient) as measured for ventricular tachycardia patients according to an exemplary embodiment of the present disclosure;

FIG. 17 is a set of exemplary panels illustrating centroid-centroid and dice coefficient values in training and test groups for atrial tachycardia patients and a comparison of the ground truth and predicted areas, and the number of predicted groups per case for the training and test groups according to an exemplary embodiment of the present disclosure;

FIG. 18 is an illustration of exemplary primary and secondary outcomes according to an exemplary embodiment of the present disclosure;

FIG. 19 is an illustration of an exemplary block diagram of an exemplary system in accordance with certain exemplary embodiments of the present disclosure; and

FIGS. 20A-20D are exemplary graphs providing an exemplary feature extraction.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

The following description of exemplary embodiments provides non-limiting representative examples referencing numerals to particularly describe features and teachings of different aspects of the present disclosure. The exemplary embodiments described should be recognized as capable of implementation separately, or in combination, with other exemplary embodiments from the description of the exemplary embodiments. A person of ordinary skill in the art reviewing the description of the exemplary embodiments should be able to learn and understand the different described aspects of the present disclosure. The description of the exemplary embodiments should facilitate understanding of the exemplary embodiments of the present disclosure to such an extent that other implementations, not specifically covered but within the knowledge of a person of skill in the art having read the description of embodiments, would be understood to be consistent with an application of the exemplary embodiments of the present disclosure.

A common mechanism of arrhythmia generation involves circus-movement rotation of electricity called reentry. (See, e.g., Refs. 18 and 19). The critical component, or isthmus, of such a circuit is typically an area of diseased tissue isolated from the rest of the myocardium by functional or anatomic barriers. Catheter ablation typically uses an energy source to create local areas of tissue necrosis intended to either isolate or eradicate the abnormal electrical activity responsible for the arrhythmia. One of the main goals of an electrophysiology procedure is to determine the location of such isthmuses, as these locations are ideal targets for ablation. However, this process may be time-consuming (see, e.g., Refs. 19 and 20), and may not be possible with transient or hemodynamically unstable arrhythmias.

Reentrant arrhythmias, such as, e.g., ventricular tachycardia (VT) and atrial tachycardia (AT), can be ablated after first creating an electroanatomic map by sampling thousands of individual points, each consisting of an electrical signal (an electrogram) and associated location information. The electroanatomic mapping system summarizes these data as a graphical representation of the activation timing or voltage recorded at each sampled point. However, interpretation of this information in order to identify an isthmus is still mainly done through visual perception, and is prone to inter-operator variability. This can contribute to the high recurrence rates in certain ablations. (See, e.g., Refs. 19-21).

According to the exemplary embodiments of present disclosure, systems, methods and computer-accessible medium can be provided to facilitate and/or utilize an exemplary procedure that predicts the location of an arrhythmogenic substrate in scar-mediated reentrant tachycardias. The exemplary systems, methods and computer-accessible medium can first derive quantitative features from conventional electroanatomic mapping data that describe known isthmus characteristics. These exemplary features can then be used to train a neural network to predict which of the points sampled during a procedure lie on a critical isthmus. A predictive algorithmic approach of the exemplary systems, methods and computer-accessible medium can facilitate a real-time substrate assessment, providing the electrophysiologists an objective tool with which to plan the ablation strategy.

The systems, methods and computer-accessible medium according to the exemplary embodiments of the present disclosure can be provided (which can include computer software) which can utilize the electroanatomic information obtained during ablation procedures to automatically extract features from the recorded signal. These exemplary features and their associated anatomical positions can be applied to a machine learning procedure which has the capacity to “learn” which features are associated with the location of critical isthmuses. This information can then be used to prospectively predict the location of isthmuses.

Thus, using the exemplary systems, methods and computer-accessible medium described herein, it is possible to provide an electrogram feature set that is unique to the reentry isthmus in ventricular and atrial tachycardias.

One object of the exemplary embodiments of the present disclosure is to identify unique electrogram (EGM) features associated with the reentry isthmus location that can aid in targeting reentrant circuits because these signals contain abnormal electrophysiologic parameters and remodeling characteristics. The exemplary systems, methods and computer-accessible medium according to exemplary embodiments of the present disclosure can retrospectively acquire EGMs from patients undergoing catheter ablation (n=475) of atrial tachycardia (AT) or ventricular tachycardia (VT). Patients with AT can be studied during AT arrhythmia. As VT is typically not hemodynamically sustained, patients with VT can be studied in their presenting (non-VT) rhythm. For example, the total combined data set can contain >2 million data points. Using the exemplary systems, methods and computer-accessible medium according to exemplary embodiments of the present disclosure, an optimal set of features can be derived from the sampled EGMs, describing aspects of voltage amplitude, conduction velocity, scar-conduction block relationships, wavefront propagation, and the shape characteristics of electrogram deflections, all of which are differentiable at isthmus versus non-isthmus sites. Areas belonging to the isthmus, scar, and healthy tissue can be summarized with descriptive statistics and an analysis of variance (ANOVA) can be conducted to assess statistical significance between the groups for each feature. The exemplary systems, methods and computer-accessible medium according to exemplary embodiments of the present disclosure can provide an optimized set of electrogram features that distinguish and can provide a unique solution for targeting the reentry isthmus.

### Localizing Reentry Isthmus in Atrial and Ventricular Tachycardia Using Deep Learning

Although advancements have been made in the computer-aided visualization of EGM information, including calculation of local conduction velocity and depiction of conduction block, isthmus identification during ablation procedures is still primarily performed by visual perception, which is prone to variation in interpretation. In preliminary studies, the feasibility of using a graph-convolutional neural network (GCN) trained on EGM features to predict the location and shape of the reentry isthmus has been demonstrated. Exemplary systems, methods and computer-accessible medium according to exemplary embodiments of the present disclosure can assess the ability of a GCN and other deep learning architectures oriented towards point-cloud input to automatically detect the reentry isthmus location using an optimized feature set. Datasets can be divided into training 70%, validation 15%, and testing 15%. Primary outcome metrics can include the Dice coefficient (a measure of area overlap) and centroid-to-centroid distance between actual and predicted isthmus. Exemplary systems, methods and computer-accessible medium according to exemplary embodiments of the present disclosure can provide a quantitative, automated, and accurate assessment of the likelihood that an EGM-sampled region will be crucial for maintaining tachycardia and therefore should be ablated.

Exemplary systems, methods and computer-accessible medium according to exemplary embodiments of the present disclosure can establish a new quantitative paradigm in electroanatomical analysis of scar-mediated reentry. The exemplary procedures developed according to exemplary systems, methods and computer-accessible medium of exemplary embodiments of the present disclosure can facilitate operators to target arrhythmogenicity more succinctly than the current standard-of-care, ultimately reducing procedure time and likely improving ablation success rates.

A common mechanism of arrhythmia generation involves circus-movement rotation of electricity called reentry. The critical component, or isthmus, of such a circuit is typically an area of diseased tissue isolated from the rest of the myocardium by conduction block or anatomic barriers (i.e., regions of scarring or a valve annulus). Such isthmuses are typically targeted during catheter ablation procedures for reentrant arrhythmias.

Ventricular arrhythmias are the largest cause of sudden death in the U.S., with an estimated 300,000-350,000 deaths per year. (See, e.g., Ref. 18). Ventricular tachycardia (VT), a type of ventricular arrhythmia, can result in sudden death if not treated promptly by terminating the rhythm, usually with an electric shock. In people with implanted defibrillators, VT can result in recurrent shocks from the device, which can be traumatic and painful (see, e.g., Ref. 22) and are sometimes delivered inappropriately. Episodes of VT can be prevented with a radiofrequency ablation procedure, which can reduce the risk of these endpoints. (See, e.g., Ref. 23). Ablation procedures to treat VT range in length from 3-6 hours in duration, with a 25%-50% long term recurrence rate (average 3 years after ablation). (See, e.g., Ref. 20).

Reentrant atrial tachycardias typically occur in patients after cardiac surgery (see, e.g., Ref. 24) or after an ablation procedure for atrial fibrillation (AF) (see, e.g., Ref. 19), with up to 24% experiencing this rhythm post-procedurally in the early AF ablation experience. (See, e.g., Ref. 25). ATs increase the risk of thromboembolic events, can cause symptoms such as shortness of breath or fatigue, and can precipitate heart failure if not adequately controlled. (See, e.g., Ref. 26). Similar to VT, ablation for AT can be prolonged, and recurrence rates can be as high as 38%. (See, e.g., Ref. 19).

### Exemplary Current Standard of Care for Ablation Using Electroanatomic Mapping

Complex arrhythmias are ablated using information obtained by creating an electroanatomic map in which thousands of individual points are sampled, each consisting of an electrical signal (an EGM) and associated location information. The electroanatomic mapping system (EAMS) summarizes these data as a graphical representation of the activation timing or voltage recorded at each sampled point. While some advancements have been made in automated data analysis including automated data binning algorithms (see, e.g., Ref. 27) and a best-fit solution algorithm that calculates regions of slow conduction or block (see, e.g., Refs. 28 and 29), such techniques still require visual operator interpretation. In clinical practice, therefore, isthmus identification is still mainly done through visual perception, and is prone to variations in interpretation. (See, e.g., Ref. 30). This can contribute to the high recurrence rates in certain ablative therapies. (See, e.g., Ref. 31).

The process of interpreting electroanatomic maps to identify targets for ablation is time-consuming and may not be possible when the arrhythmia in question is transient or would be dangerous to the patient if sustained. Techniques have been developed to predict isthmus locations from information obtained when the patient is not in arrhythmia, such as associating isthmus locations with changes in local conduction velocity properties (see, e.g., Ref. 11) and identifying VT exit sites by reproducing the clinical VT ECG morphology with pacing. (See, e.g., Ref. 32). Still, even with these techniques, the overall arrhythmia-free success rate of many ablations remains in the 50-70% range. (See, e.g., Refs. 19, 20, 31). Procedure times are long for most ablations (see, e.g., Ref. 33), limiting the number of arrhythmias that can be addressed in one procedure and increasing the risk of procedure-related complications.

Exemplary Isthmuses in VT patients studied in VT: Studies of patients in VT have established that VT isthmus regions contain low-amplitude signals typically measured during electrocardiogram segments that are otherwise electrically silent (diastole). (See, e.g., Ref. 5). High-density mapping studies have shown that conduction velocity is relatively preserved in isthmus zones. In contrast, regions preceding and following the isthmus have comparatively slower velocities (see, e.g., Ref. 3), typically accompanied by longer-duration electrograms. (See, e.g., Ref. 7). Studies of post-infarction VT have established that isthmuses are composed of regions of viable tissue in otherwise damaged areas, identified by voltage measurement as scar. (See, e.g., Ref. 34). Electricity typically travels around an anchor of anatomic or functional electrical block and reenters the isthmus area (see, e.g., Ref. 3, 4), traversing the isthmus before exiting, typically in the border zone between normal myocardium and scar.

Exemplary Electrical properties of VT isthmuses studied in sinus rhythm: Substrate ablation (ablation of abnormal areas of myocardium during a non-VT rhythm) is commonly performed when treating ventricular tachycardia (VT) since VT is frequently too hemodynamically unstable to map adequately. Ablation is ideally aimed at the central component of VT isthmuses, but isthmus components can only be determined by mapping during VT. It can be optimal to establish the electrogram characteristics of VT isthmuses and their components in a patient's baseline, non-VT rhythm to improve the accuracy of lesion delivery. Early work characterizing sinus EGMs in VT patients identified decreased signal amplitude, increased fractionation, and the presence of double potentials as characteristics seen in isthmus regions. (See, e.g., Ref. 35). Canine post-infarction studies have shown that in sinus rhythm, isthmus areas display uniform, slow conduction. (See, e.g., Ref. 36). Noncontact mapping studies of VT patients in sinus rhythm have suggested distinctive EGM width in isthmus locales versus the periphery of the border zone. (See, e.g., Ref. 37). Regions of slow conduction, visualized with high density mapping as regions of isochronal crowding, have been seen to correspond to critical sites for VT propagation (see, e.g., Ref. 10), the ablation of which is associated with freedom from clinical VT in humans. (See, e.g., Ref. 11). When assessed in sinus rhythm, isthmuses have been noted to display biphasic, short-duration signals in comparison to surrounding signals. (See, e.g., Refs. 8, 9). While the detailed architecture and EGM characteristics of isthmuses densely mapped during VT have been described (see, e.g., Ref. 38) more data is required on the nature of non-VT EGM features in VT patients, and how these characteristics might vary between isthmus components.

Exemplary Isthmuses in atrial tachycardias (ATs): Many of the concepts used to describe VTs apply to ATs as well. AT isthmuses in patients with atrial scarring were identified during the early history of arrhythmia ablation. (See, e.g., Ref. 12). More recently, isthmus locations and characteristics of ATs in patients with prior ablation for atrial fibrillation have been described. (See, e.g., Ref. 13). The development of high-density mapping, along with the fact that ATs are generally better tolerated than VTs, has allowed for the detailed characterization of AT isthmus properties. (See, e.g., Refs. 28, 29, 14). Such high-density mapping studies have shown atrial isthmuses to be associated with lower voltage than non-isthmus regions. (See, e.g., Ref. 14). In contrast to VTs, conduction velocity in atrial isthmus areas is decreased compared to entry or exit regions. (See, e.g., Ref. 14). Other electrogram features have proven useful, such as the presence of double-potentials, which have been shown to correspond to areas of conduction block and would be expected to be adjacent to isthmus locations. (See, e.g., Ref. 15).

Exemplary Summary of Machine Learning and Deep Learning within Cardiac

### Electrophysiology

Modern data science technology has emerged as a mechanism for quickly collating, analyzing, and processing large amounts of data. Machine learning is a branch of data science that allows a computer to determine empirical relationships between data variables, which can then be applied to predict new data that the system will encounter. Machine learning has been used extensively with surface ECG recordings for arrhythmia classification, (see, e.g., Refs. 39-40) and in medical imaging for anatomic segmentation (see, e.g., Refs. 41 and 42), but has been used only once in the analysis of intracardiac electrograms, for guidance of ablation for atrial fibrillation. (See, e.g., Ref. 43).

### Exemplary Unmet Need

Although recent years have seen advances in ablation technology, there remains considerable room for improvement of procedural efficiency and outcomes. The duration of ablation procedures for VT and complex ATs remains long, limiting the number of arrhythmias that can be addressed in one procedure and increasing the risk of procedure-related complications, and the recurrence rates are high. (See, e.g., Refs. 19, 20, 44). Morbidity can remain high for patients with recurrences, especially for those that remain at risk of sudden death and/or of defibrillator shocks, which can be traumatic and painful. While a great deal of information is available in high-density electroanatomic maps, the interpretation of this information is largely done through visual interpretation of a limited number of EGM features. The reliance on largely manual interpretation of such a large amount of data is challenging for the operator, and inevitably leads to the discarding of much information. As an important isthmus of a reentrant arrhythmia is generally a small region, errors in data acquisition or interpretation can lead to the failure of the operator to adequately identify and eliminate a critical isthmus, as well as result in imprecise ablation of areas of the heart not critical for arrhythmia maintenance, thus causing unnecessary damage and creating the substrate for additional arrhythmias.

Thus, there is an important need to facilitate a more efficient characterization of arrhythmia circuits and their substrate by intelligently predicting the location of critical isthmuses, thereby improving the quality and efficiency of cardiac arrhythmias therapy. The exemplary systems, methods and computer-accessible medium according to exemplary embodiments of the present disclosure can identify electrogram features of isthmuses in patients with VT and AT and develop predictive algorithms to meet this need.

Utilizing quantitative information from electrograms: As described herein, EGM amplitude, duration, number of deflections, and conduction velocity have been assessed in patients during AT and VT. (See, e.g., Ref. 3). However, much of the data obtained for patients in VT has been obtained in patients during VT (see, e.g., Ref. 3), or prior to the era of high-density mapping, whereas VT ablation today is most commonly performed when patients are in their baseline, non-VT rhythm. High density mapping to identify regions of isochronal crowding has been used as a surrogate marker for the VT isthmus location, but this approach is qualitative, and relies on the operator's visual interpretation of isochronal density to choose an ablation target. Exemplary systems, methods and computer-accessible medium according to exemplary embodiments of the present disclosure provide a quantitative description and visualization of currently utilized EGM features, allowing for a more objective assessment of arrhythmia substrate.

Unique features for identifying isthmus regions within tachycardia and sinus rhythm: Modern data science computational techniques allow for the rapid analysis of a large volume of data in a short time. While several EGM characteristics have been used to identify critical ablation targets as described above, their relative utility has yet to be quantified. Additionally, the scope of EGM features previously used for ablation target selection remains limited to basic characteristics primarily evident by visual inspection. A more quantitative and comprehensive assessment of novel EGM features according to exemplary systems, methods and computer-accessible medium according to exemplary embodiments of the present disclosure can provide operators a more extensive, better-defined set of tools to accurately determine sites for ablation.

Machine and Deep Learning for identifying ablation targets: A machine learning-aided approach to complex ablation according to exemplary systems, methods and computer-accessible medium according to exemplary embodiments of the present disclosure is a novel precision-medicine tool, a methodology for efficiently identifying the small but critical area of the heart required for arrhythmia maintenance. This can improve the frequently imprecise ablation methods currently employed, potentially improve ablation success rates, and decrease procedure times. Since complex ablations are presently done only by high-volume institutions with sufficient throughput to achieve manual expertise, machine-assisted selection of ablation targets can allow additional operators to perform ablation, potentially expanding access to this vital therapy to patients not currently receiving it.

GCNs for isthmus detection: In exemplary systems, methods and computer-accessible medium according to exemplary embodiments of the present disclosure, both supervised and unsupervised machine learning techniques can be used for data classification. While unsupervised techniques such as k-means clustering do not require time-consuming manual labeling, unsupervised clustering can be sensitive to outlier data, may not typically perform well in large, high dimensional data sets, and may not yield groupings relevant to the desired application. A trained deep learning network (DLN) is a supervised machine learning technique with the potential for non-linear real-time analysis of a large volume of complex data. It can be a more appropriate tool when target classification is narrowly defined. (See, e.g., Refs. 45 and 26). A GCN (a subtype of DLN) is a well-defined framework that uses graph connections to process irregularly spaced three-dimensional data generated by electroanatomic mapping systems. (See, e.g., Ref. 47). Other mechanisms for analyzing such information, such as two-dimensional transformation, have been used but are susceptible to spatial distortion. (See, e.g., Ref. 48). Three-dimensional convolution has been implemented for imaging analysis (see, e.g., Ref. 49), although it assumes regularly spaced data and requires significantly more computation than is strictly necessary for points confined to a two-dimensional surface. Exemplary systems, methods and computer-accessible medium according to exemplary embodiments of the present disclosure can use a graph-based architecture via graph connections which can allow EAMS data to be processed by the DLN while preserving its spatial integrity and avoiding interpolation.

Mapping catheters with many electrodes (e.g., 16-48) have provided higher-resolution maps, leading to shorter ablation procedures and improved patient outcomes. (See, e.g., Refs. 23, 50). These maps, with thousands of points, significantly increase the spatial resolution of the reconstructed map, enabling electrophysiologists to more precisely identify substrates for ablation. Exemplary systems, methods and computer-accessible medium according to exemplary embodiments of the present disclosure provide advancements of this process by training a machine learning model to identify critical arrhythmia isthmus points. This can revolutionize how the medical community approaches ablation procedures, leading to more targeted and efficient treatments and, ultimately, better patient outcomes.

Experimental dataset from consented patients: The dataset used in exemplary systems, methods and computer-accessible medium according to exemplary embodiments of the present disclosure can be obtained through both prospective and retrospective analysis of mapping procedures at a medical facility (e.g., at Columbia University Irving Medical Center CUIMC). Under an IRB-approved protocol (IRB #AAAT8818) patients who have undergone clinical ablations for either AT or VT performed with either the CARTO (Biosense Webster), or the Ensite Precision or Ensite X (Abbott Medical) mapping systems can be enrolled in a database. Of these patients, those with scar-related reentry can be included.

The mapping systems and catheters currently available include, for example, the CARTO 3 EAMS and a multi-spline catheter (PENTARAY; 20-poles with 3 mm spacing or Octaray; 48-poles with 2 or 3 mm spacing, Biosense Webster, Diamond Bar CA) or the Ensite X EAMS and a grid-shaped 16-pole catheter with 4 mm spacing (HD Grid; Abbott Cardiovascular, Plymouth MN). Ablation can be performed using, e.g., either the SmartTouch SF (Biosense) or TactiCath (Abbott Cardiovascular) irrigated radiofrequency ablation catheters.

Exemplary Sample size. Exemplary systems, methods and computer-accessible medium according to exemplary embodiments of the present disclosure can utilize a sufficient sample size, e.g., 475 cases, including those mapped for N=225 AT ablation and N=150 mapped for VT ablation. Based on previous experience, it can be estimated that for VT cases, maps can be created in the presenting non-VT rhythm for all cases, and in VT in about 10% of these cases. Exemplary systems, methods and computer-accessible medium according to exemplary embodiments of the present disclosure have identified 38 AT and 40 VT cases acceptable for analysis, among cases done within the prior 18-month period (using the current software versions of the CARTO and Abbott systems and representing approximately 700 procedures). Additional cases can be selected from the ablation procedures performed at CUIMC during the study.

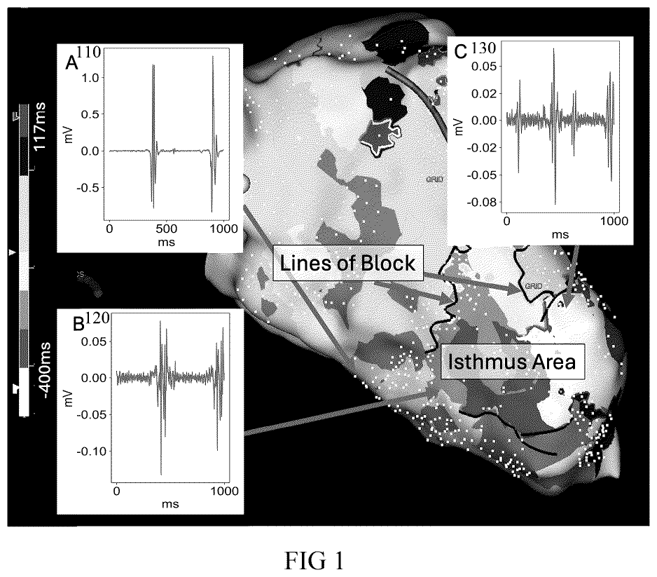

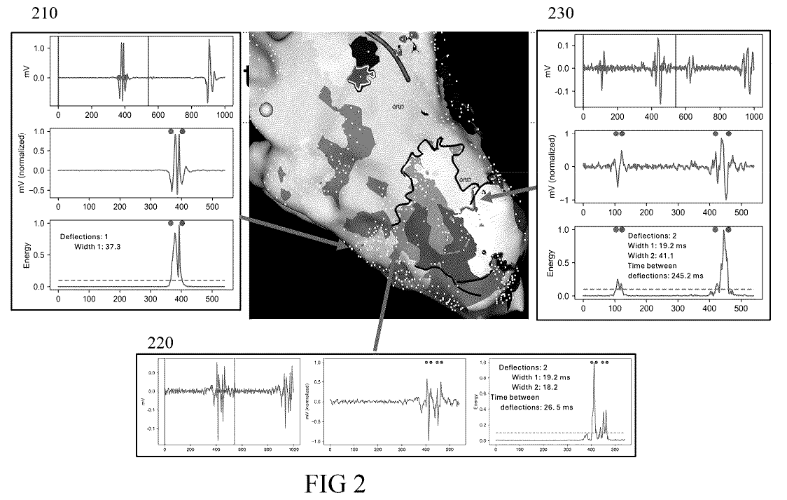

Labeling and Identification of the Isthmus and Lines of Block. For patients mapped during arrhythmia, exemplary systems, methods and computer-accessible medium according to exemplary embodiments of the present disclosure can define isthmuses as regions of abnormal voltage (e.g., <1.5 mV) and conduction velocity (e.g., <1.0 cm/sec) bounded by one or more lines of conduction block, regions of scar, and/or anatomical boundary through which a reentrant impulse passes for tachycardia propagation. Exemplary FIGS. 1 and 2 illustrate this point.

FIG. 1 shows an example of electrograms taken from different sections of a left ventricle during an atrial tachycardia ablation. Shades represent the order of electoral activation as seen in the legend. Each sampled point is represented by a dot. Solid black lines were drawn during post-procedure review and represent lines of conduction block. The isthmus region is adjacent to scar and bounded by lines of block. In FIGS. 1-3, graphs 110, 210 and 310 show a relatively healthy electrogram far from the critical arrhythmia location. Graphs 120, 220 and 320 indicate an electrogram near the exit of the critical region. 130, 230 and 330 show an electrogram near the entrance to the critical region. In each of graphs 210, 220, 230, 310, 320 and 330, the top graph shows the unprocessed recorded electrogram, the middle graph is a normalized electrogram representing one cardiac cycle, and the bottom graph is the single cardiac cycle electrogram processed with an instantaneous energy operator in order to detect individual electrogram deflections. In these graphs, leading dots represent the detection of a deflection onset and trailing dots deflection offset, while the dotted line represents the threshold energy above which a complex is recorded. Scar can be defined as voltage of <0.05 mV. (See, e.g., Refs. 28, 29 and 14). Lines of block can be defined as areas with apparent conduction velocity below the threshold of what is physiologically possible. The isthmus start can be defined as the area where the wavefront curvature first coalesces into this region, and its terminus as the area at which constrained wavefront conduction ceases and breakout to the remainder of the myocardium occurs. (See, e.g., Ref 38). Isthmus subcomponents can be defined by dividing in equal sections the shortest path through the isthmus into entrance, middle or central, and exit sections in order of activation during AT/VT. An adjacent bystander can be defined as an area electrically contiguous with the isthmus but activated passively during AT/VT. An inner and outer loop can participate in the circuit but can be distal to the central component and are either protected (e.g., inner loop) or unprotected (e.g., outer loop) by surrounding scar. In addition to detailed activation mapping, isthmus components can be identified with response to entrainment when available. (See, e.g., Ref. 51).

With the exemplary systems, methods and computer-accessible medium according to the exemplary embodiments of the present disclosure, for VT patients mapped in their presenting non-VT rhythm, surrogate markers can be used to identify critical areas capable of acting as isthmuses. Such markers can include voltage, isochronal late activation mapping (ILAM), (see, e.g., Ref. 11) and identification of regions that when paced, demonstrate electrical latency followed by an EGM morphology equal to that seen in the clinical VT. (See, e.g., Ref. 32). Clinical VT morphology can be obtained from prior ECGs of the VT, EGMs of transiently induced VT during the ablation procedure, or from tracings recorded from an implanted defibrillator. In exemplary systems, methods and computer-accessible medium according to exemplary embodiments of the present disclosure, all isthmuses and sections can be identified, and mechanisms confirmed in off-line analysis by the consensus opinion of three investigators (DS, EC, and EW).

Registration of isthmus and components to non-VT maps: According to exemplary systems, methods and computer-accessible medium according to exemplary embodiments of the present disclosure, a subset of data in VT patients can include both the VT and non-VT maps created using the same underlying geometry. Points in non-VT maps within 1 mm of VT isthmus points can be assigned an isthmus label.

### Electrogram Feature Set Unique to the Reentry Isthmus in Ventricular and Atrial Tachycardias

Ablation of scar related reentrant arrhythmias can require identification of a critical isthmus. While AT is usually well tolerated and ablated in tachycardia, VT can be hemodynamically unstable, and ablation is commonly done in the presenting (usually sinus) rhythm. Identifying unique EGM features associated with the known reentry isthmus location can aid in subsequently targeting reentrant circuits, because these signals reflect abnormal electrophysiologic parameters and remodeling characteristics.

### Exemplary Data

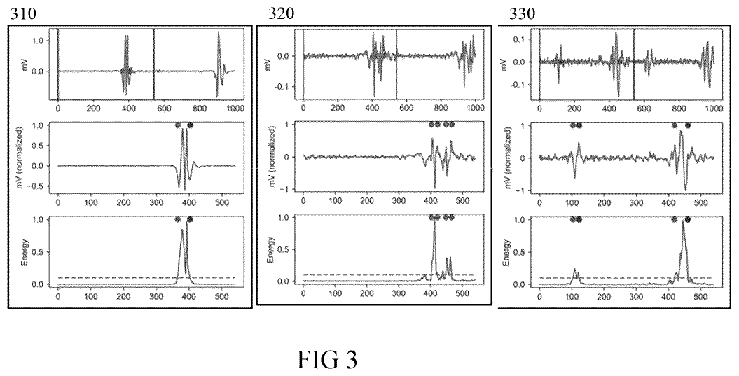

Preliminary electrogram characteristics of isthmuses in tachycardia and in sinus rhythm: Exemplary systems, methods and computer-accessible medium according to exemplary embodiments of the present disclosure provide for analysis of EGM features in 29 patients with AT (e.g., mean age 63.5 years, 27.8% female) and 7 patients with VT (e.g., all men, median age 64.0 (10.0) years) from among patients in the preliminary dataset (see FIG. 3-5). In this initial analysis according to the exemplary embodiments of the present disclosure, all VT patients had electroanatomic maps in both VT and a baseline paced or conducted (non-VT) rhythm available. For AT patients, a total of 193,821 points were collected. For VT patients, 16,433 points were collected in the non-VT rhythm and 11,701 in VT. Basic EGM features, such as amplitude, activation time, and location can be exported directly from the mapping software. Custom features can be calculated from the exported EGM signal, including the percentage-wise duration of electrical activation, the distance from the point to areas of scar or conduction block, signal power, conduction velocity (CV) and vector calculated by trigonometric triangulation (see, e.g., Ref. 16), mean regional conduction speed (MRCS; untriangulated conduction speed measured between adjacent point pairs), and the mean angle between a point's conduction vector and surrounding vectors (see, e.g., Table 1).

TABLE 1
Exemplary List of features used for network training.

| | |
|---|---|
| Minimal Regional | Signal Power |
| Conduction Speed | |
| (MRCS) | |
| Distance to scar | Signal Percentage |
| Distance to block | Number of Deflections |
| Distance to barrier | Percent vector |
| | incoherence |
| Double potential | Forward maximal LAT |
| spacing | Difference (FMLATD) |
| Conduction velocity | Peak Frequency |
| (CV) | |
| Voltage | Maximum Deflection |
| Mean Vector Angle | Width of Maximal |
| | Amplitude Deflection |
| LAT Range | Total Signal Width |

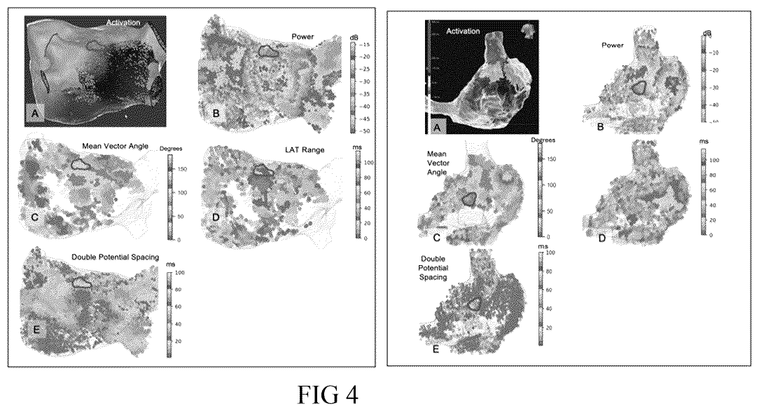

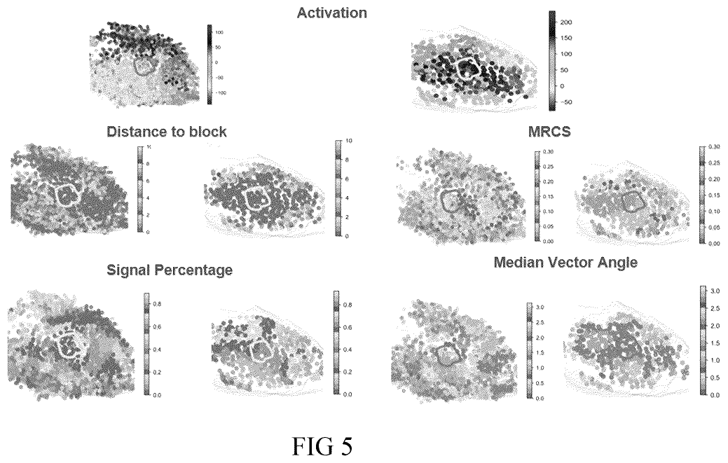

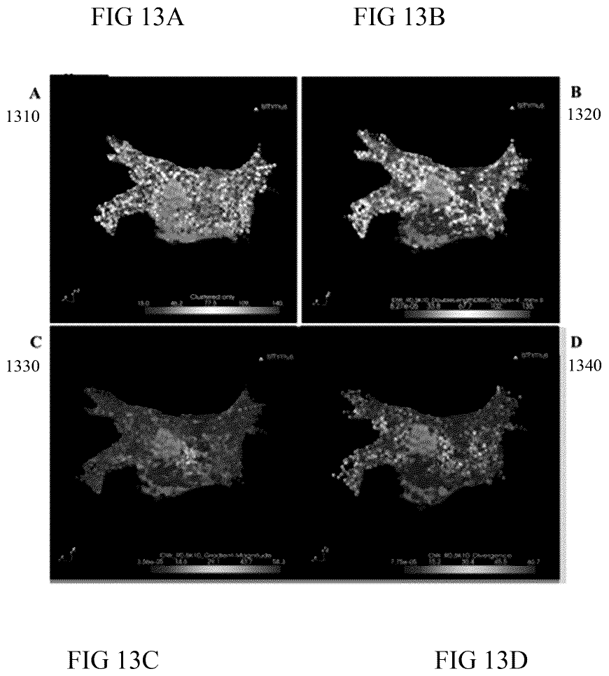

Exemplary maps of many of these features are shown in FIGS. 4 and 5. For example, FIG. 4 shows two mapped atrial tachycardias with visual representations of their feature values. For both right and left panels, features shown are A) activation, B) power C) mean vector angle, D) LAT range, and E) spacing of double potentials. Isthmus are circled. In FIG. 5, exemplary maps are shown from a single patient mapped in both sinus rhythm (left) and VT (right). Isthmuses are circled. In exemplary systems, methods and computer-accessible medium according to exemplary embodiments of the present disclosure, isthmus areas can be identified in all cases mapped in tachycardia. For non-VT rhythm mapped VT cases, isthmus locations can be transferred by registering their locations using the map created in VT. FIG. 13 provides exemplary visualizations of features extracted from electroanatomic maps from AT, where (A) is original double potential values, (B) is double potential values interpolated back from the regular grid after processing, (C) is visualization of the double potential gradient and (D) is divergence.

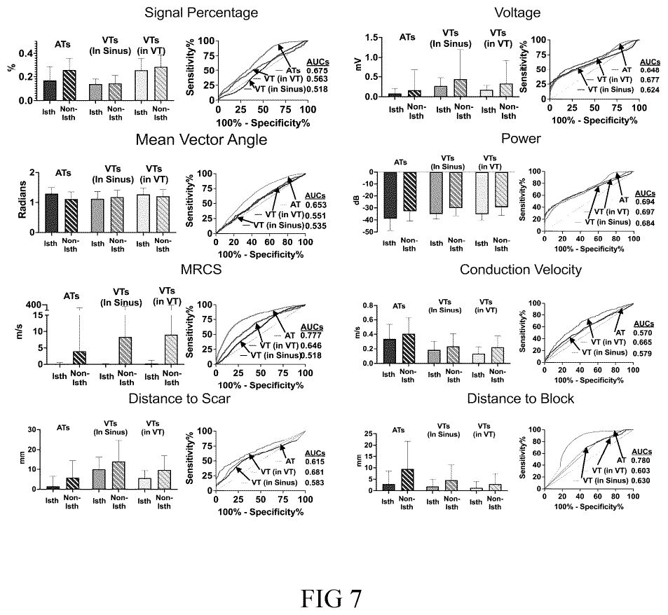

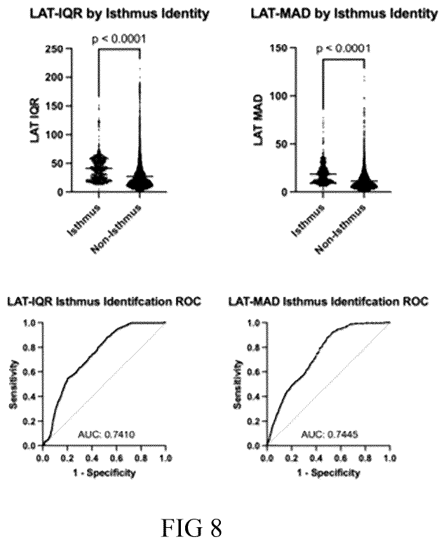

Using the exemplary systems, methods and computer-accessible medium according to the exemplary embodiments of the present disclosure, significant differences between feature values in isthmus and non-isthmus regions can be seen in all rhythms with the exception of signal percentage measured for VTs mapped in a non-VT rhythm. The strength of the association of each feature with isthmus identity can be calculated, as reflected in exemplary illustrations of FIG. 7, and can be found to vary with rhythm. As shown in FIG. 7, eight example features shown in 3 different rhythms (atrial tachycardia (AT), ventricular tachycardia patients measured in sinus rhythm (VTs in sinus), and ventricular tachycardia patients measured in VT (VTs in VT). For each feature, the left panel shows the values of the features in each rhythm for among isthmus (Isth) and non-isthmus (Non-Isth) points. The right panels show the receiver-operator curves describing the ability of the given feature in distinguishing isthmus from non-isthmus points. AUC is area is under the curve. For example, signal percentage, MRCS, mean vector angle, and distance to block can display the largest areas under the receiver operating characteristic curve (AUC) values for AT cases. However, according to exemplary systems, methods and computer-accessible medium according to exemplary embodiments of the present disclosure, AUCs for distance to scar, conduction velocity, and voltage can be highest among VT patients. Power can perform similarly in all rhythms. In general, AUCs can be lowest for VTs mapped in a non-VT rhythm, highlighting the need for novel feature generation in this rhythm. FIG. 8 illustrates, for ventricular tachycardia patients studied in sinus rhythm, the measurement of local activation time (LAT) interquartile range (IQR) and LAT median absolute deviation (MAD) in isthmus and non-isthmus points (top panels) and the receiver-operator curves (ROC) for these measurements in distinguishing isthmus from non-isthmus points.

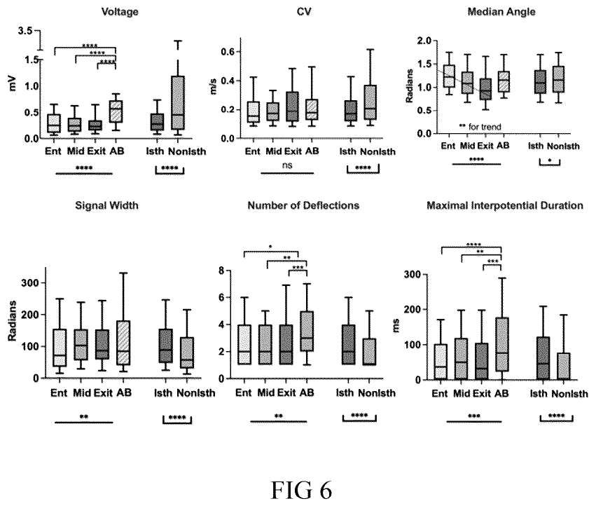

Exemplary data on differences in electrogram characteristics among components of the VT isthmus mapped in a non-VT rhythm: According to exemplary systems, methods and computer-accessible medium according to exemplary embodiments of the present disclosure, among the seven VT patients, voltage, conduction velocity, signal width, mean angle between conduction vectors, number of deflections, and the maximum duration between multicomponent EGMs can be recorded and compared between isthmus and non-isthmus regions as well as between isthmus subcomponents as defined above. as compared to non-isthmus areas, isthmus areas mapped in the non-VT rhythm can display a greater number of EGM deflections, a lower CV, a smaller median vector angle between conduction vectors, longer signal width, decreased voltage, and increased maximal interpotential duration. Exemplary FIG. 6, illustrates the performance of six features in ventricular tachycardia patients measured in sinus rhythm. Feature values for isthmus points (Isth) are compared to non-isthmus points (Non-Isth). Entrance (Ent), middle (Mid), exit (Exit), and adjacent bystander (AB) sections of the isthmus are compared to each other. Groups are compared using non-paramteric analysis of variance. Pairwise comparisons are done using Mann-Whitney tests. **** p<0.0001 *** p<0.001 ** p<0.001 *p<0.05 ns=not significant. In exemplary systems, methods and computer-accessible medium according to exemplary embodiments of the present disclosure, there can be significant differences in non-VT features between isthmus components in all measures assessed except for CV. There can be a statistically significant trend towards progressively smaller median vector angle proceeding from isthmus entrance to exit (e.g., p<0.001). Adjacent bystanders can display intermediate values. Voltage can be significantly higher in adjacent bystander areas than in entrance, middle, or exit sections of the isthmus in pairwise comparison (e.g., p<0.0001 for all comparisons). According to exemplary systems, methods and computer-accessible medium according to exemplary embodiments of the present disclosure, adjacent bystander areas can also display a greater median number of EGM deflections and increased maximal interpotential duration than other isthmus components.

Double Potential Gradient within Critical Isthmuses of Scar Related Atrial Tachycardia: Double potentials (DP) are EGM features that display two discrete activity complexes in the same cardiac cycle. Widely spaced potentials can be frequently seen when the recording catheter lies across a line of conduction block. When conduction occurs across a region with conduction delay, as may be seen in the region of an isthmus, the duration of baseline between potentials varies directly with the distance of the recording catheter to the region of breakthrough. Exemplary systems, methods and computer-accessible medium according to exemplary embodiments of the present disclosure can assess the ability of various DP measurements to localize the isthmus in the cohort of 29 AT patients. Since electroanatomic point clouds are irregular, DP gradients can be calculated by interpolating to a regular grid via inverse distance weighting. Gradients can then be back-interpolated to the original point cloud for analysis. In exemplary systems, methods and computer-accessible medium according to exemplary embodiments of the present disclosure, pooled isthmus points can have higher DP gradient values than non-isthmus points (e.g., Mann-Whitney, p<0.001). On logistic regression, the DP gradient can be more predictive of isthmus location than unmodified DP spacing (e.g., AUC=0.714 vs AUC=0.580, DeLong test p<0.001.), suggesting that the DP gradient is a more accurate predictor of isthmus points than DP and may be a useful feature in the identification of isthmus points.

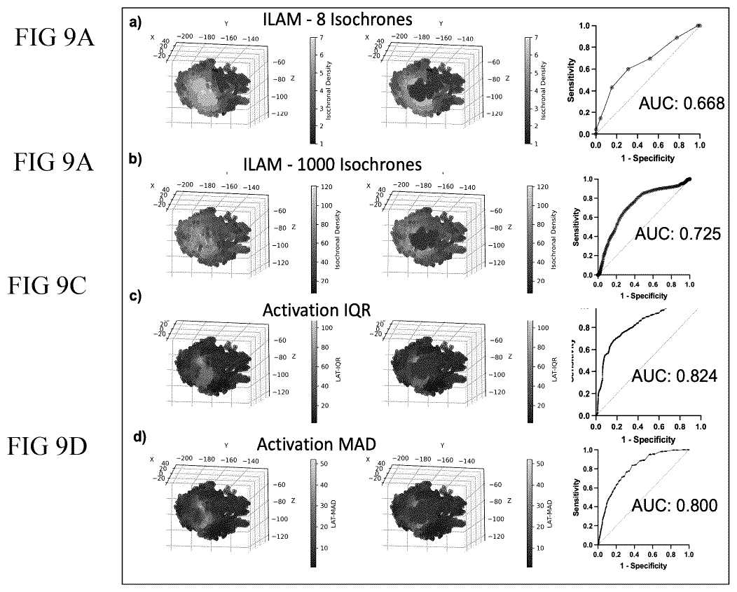

Assessment of quantitative and alternative measures of isochronal mapping: In VT ablations, ILAM is frequently used to identify regions of isochronal crowding, which have been seen to co-localize with the VT isthmus. (See, e.g., Refs. 2, 52). This strategy involves binning sampled points into 8 isochrones and visually identifying areas in which 3 or more isochrones are present in a 1 cm radius. This approach is inherently qualitative, is limited in spatial resolution, and is susceptible to inaccuracies due to outlier values. Isochronal crowding can, however, be visualized as a quantitative metric. According to exemplary systems, methods and computer-accessible medium according to exemplary embodiments of the present disclosure, increasing the number of binned isochrones raises the predictive ability isochronal density. Exemplary FIG. 9 illustrates this point. In FIG. 9 includes quantitative visualization of ILAM maps with (a) 8 isochrones and (b) 1000 isochrones, (c) interquartile activation range and (d) mean absolute deviation of activation timing. Alternative measures of semiquantitative conduction velocity, such as the interquartile range and mean absolute deviation of activation times, may similarly be quantitatively visualized in exemplary systems, methods and computer-accessible medium according to exemplary embodiments of the present disclosure, and may perform better than the standard assessment.

Electrogram feature calculations: In the exemplary systems, methods and computer-accessible medium according to exemplary embodiments of the present disclosure, point location, local activation time (LAT), voltage, and EGM at the time of acquisition for each utilized point collected can be retrospectively exported from the mapping system. From the exported EGM, the features described above and in Table 1 can be calculated. For each point in the map, a single cycle length window can be applied to the exported EGM. The onset and offset of each activity complex in the EGM can be recorded after processing the EGM with an instantaneous energy operator (see, e.g., Ref. 17) in a manner adapted from Oesterlein et al (see, e.g., Ref. 15) (see, e.g., FIGS. 3: 310, 320 and 330, bottom plot). Using this information, exemplary systems, methods and computer-accessible medium according to exemplary embodiments of the present disclosure can record the total width of all complexes and the number of complexes identified. For multicomponent signals, exemplary systems, methods and computer-accessible medium according to exemplary embodiments of the present disclosure can record the maximal interpotential duration between complexes. For EGMs with two deflections, this measurement is equivalent to the duration between DPs. Deflections whose continuity is divided by the cycle length window can be combined and considered as a single complex. The conduction velocity vector can be calculated using trigonometric triangulation. (See, e.g., Ref. 16). With the exemplary systems, methods and computer-accessible medium according to the exemplary embodiments of the present disclosure, for each point, adjacent non-linear point pairs in a 4 mm radius (its local neighborhood) can be used and the median result recorded. The neighborhood radius can be chosen to include sufficient adjacent points for calculation while maintaining local accuracy.

Using the exemplary systems, methods and computer-accessible medium according to the exemplary embodiments of the present disclosure, conduction velocity and voltage amplitude can also be assessed using omnipolar calculations (see below). As adjacent points that lie on opposite sides of a line of conduction block can be close together, but display large differences in activation time, untriangulated conduction speeds between such pairs can be unphysiologically low. With the exemplary systems, methods and computer-accessible medium according to the exemplary embodiments of the present disclosure, setting the lower limit of conduction velocity at, for example, 0.08 m/s (see, e.g., Ref. 53), adjacent neighbors with untriangulated conduction speed below this value can be considered to lie across a line of block and can be excluded from the triangulation calculation. An adaptation of this approach to identifying regions of conduction block is in current commercial use. (See, e.g., Ref. 54). The median angle between the index vector and the neighborhood vectors can be calculated and used as a measure of vector coherence. The peak frequency of the EGM can be calculated using the Fast Fourier Transform. The distance from each point to an anatomical barrier (e.g., such as a valve), to a scar, or to conduction block can be calculated with the exemplary systems, methods and computer-accessible medium according to the exemplary embodiments of the present disclosure. Geodesic distances can be used in all calculations.

Double Potential Gradient Calculation: Using the exemplary systems, methods and computer-accessible medium according to the exemplary embodiments of the present disclosure, to gain information regarding the spatial relationships of DP spacing in relationship to the isthmus, several metrics regarding double length gradient structure in the 3D interpolated grid can be calculated. Exemplary Gradient: The gradient of the DP scalar field can be computed to obtain the direction and magnitude of the maximum rate of increase of the field, where f is the scalar DP spacing field, grad f=∇f. Exemplary Divergence: For gradient vector field F, divergence is a scalar field describing the volume density/flux of a vector field, defining how much a vector field behaves like a source (e.g., producing vectors) or a sink (e.g., absorbing vectors) at a given point. This is given equationally by div F=∇·F. Curl: The curl vector representing the local rotational behavior of a vector field is denoted curl F=∇×F. Exemplary Laplacian: The Laplacian of a scalar function is the divergence of its gradient. It measures the difference between the average value of f over the neighborhood of a point and its value at that point. If the Laplacian is positive, f has a local minimum, and if negative, f has a local maximum. Laplacian is calculated as F=∇²f=∇·∇f. The values derived above will then be interpolated back onto the original point cloud to confirm fidelity of the workflow and for assessment of ability to predict isthmus identity.

Exemplary Omnipolar mapping-derived features: Electrogram voltage traces recorded by electroanatomic mapping systems can be either unipolar (e.g., using one roving and one fixed reference electrode) or bipolar (e.g., using two roving electrodes in close proximity) measurements. Unipolar measurements can be undesirably affected by activity far from the roving electrode. Bipolar measurements reflect more localized activity, but can depend on the relative orientation of the electrodes and activation wavefront, a phenomenon known as “bipolar blindness.” (See, e.g., Ref. 55). These artifacts can impact determinations of local activation time, conduction velocity, and voltage amplitude. The so-called omnipolar mapping technique was developed by Deno et al (see, e.g., Ref. 56) to overcome these limitations, using three or more local electrodes to estimate local wavefront propagation direction, as well as “virtual” bipolar voltage along this direction. Through omnipolar mapping, exemplary systems, methods and computer-accessible medium according to exemplary embodiments of the present disclosure can estimate conduction velocity without relying on determinations of local activation time.

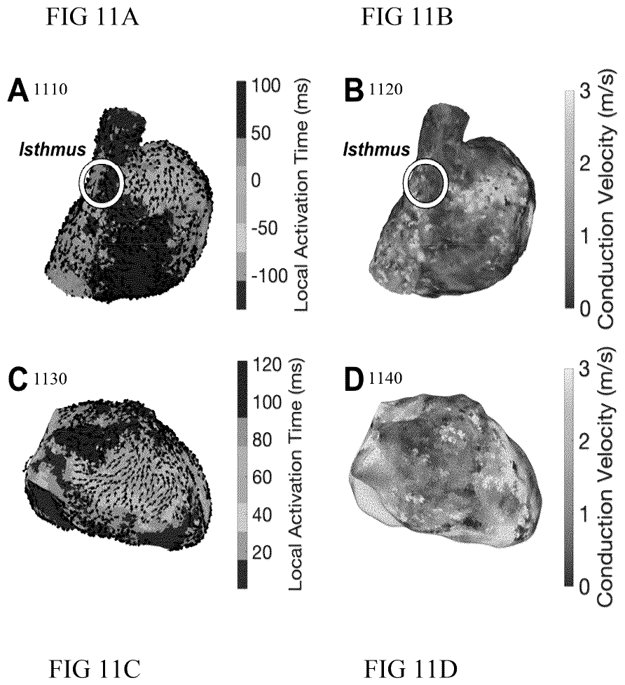

For example, exemplary omnipolar-derived features are shown on an atrial map 1110, 1120 (see FIGS. 11A and 11B) and ventricular (maps 1130, 1140 (see FIGS. 11C and 11D. In particular, FIGS. 11A and 11C illustrate omnipolar-derived activation direction vectors plotted on top of colored isochrones, and FIGS. 11B and 11D show omnipolar-derived conduction velocity maps. These conduction velocity estimates may not depend on any annotations of local activation time. True isthmus locations are circled for the atrial case. Omnipolar mapping has also been shown to be particularly advantageous for localization of isthmuses in VT. (See, e.g., Ref. 57). Omnipolar calculations can be used in exemplary systems, methods and computer-accessible medium according to exemplary embodiments of the present disclosure to obtain robust measures of activation direction, conduction velocity, and bipolar amplitude. Inclusion of these features in exemplary models according to exemplary embodiments of the present disclosure can yield better isthmus discrimination through insensitivity to system artifacts. The exemplary systems, methods and computer-accessible medium according to the exemplary embodiments of the present disclosure can be configured to apply this approach to data obtained with both grid-electrode catheters (which has been demonstrated) and deformable multi-spline mapping catheters (which has not yet been demonstrated).

Exemplary Automated Feature Extraction from Electrograms.

The exemplary systems, methods and computer-accessible medium according to the exemplary embodiments of the present disclosure can provide methods for automated feature extraction from electrograms that are robust and consistent between patients and across electroanatomical system platforms. The exemplary systems, methods and computer-accessible medium according to the exemplary embodiments of the present disclosure can provide traditional/existing methods of dimensionality reduction, including singular value decomposition (SVD) and principal component analysis (PCA). However, given the complexity of electrogram data, the exemplary systems, methods and computer-accessible medium according to the exemplary embodiments of the present disclosure can also provide methods of dimensionality reduction via deep learned representations, that may be discovered through examination of the latent space created by each model. For example, both PCA and SVD can be used as preprocessing prior to the deep learning model. To help tease out the most efficient features exemplary systems, methods and computer-accessible medium according to exemplary embodiments of the present disclosure can include “squeeze and excite” modules (see, e.g., Ref. 58) in the DLN model.

One way to visualize the ability of the model to discover and discriminate between the parameters contributing to salient electrogram features can be to map the latent space into a t-SNE (see, e.g., Ref. 59) representation/map. t-SNE (t-distributed Stochastic Neighbor Embedding) is an unsupervised non-linear dimensionality reduction technique for visualizing high-dimensional data. The t-SNE procedure seeks to find similarity measures between pairs of features in higher and lower dimensional space. It can then attempt to optimize the two similarity measures. This optimization process allows the creation of clusters and sub-clusters of similar data points that are projected into the lower-dimensional space and allow exemplary systems, methods and computer-accessible medium according to exemplary embodiments of the present disclosure to visualize and understand the structure and relationship in the higher-dimensional data. The use of such a non-linear dimensionality reduction method can help exemplary systems, methods and computer-accessible medium according to exemplary embodiments of the present disclosure discover key 2-D and 3-D clusters of features that may be hidden (not obvious) within the latent space of the DL model.

### Exemplary Statistical Analysis

The exemplary systems, methods and computer-accessible medium according to the exemplary embodiments of the present disclosure can provide and/or utilize exemplary features summarized by class (isthmus, scar, and healthy tissue) with mean, standard deviation, and an analysis of variance (ANOVA) conducted to assess statistical significance between the groups for each feature. This analysis can be carried out separately for the atrial and ventricular datasets. In addition, the exemplary systems, methods and computer-accessible medium according to the exemplary embodiments of the present disclosure can determine if features are significantly different between atrial and ventricular datasets. This can assist to inform if the exemplary systems, methods and computer-accessible medium according to the exemplary embodiments of the present disclosure can provide at least one classification model or chamber-specific models are needed in Aim 2. In addition, exemplary systems, methods and computer-accessible medium according to exemplary embodiments of the present disclosure can construct a correlation coefficient matrix to ensure that each feature provides unique information. If features are determined to be non-normally distributed, differences between groups can be tested with non-parametric Mann-Whitney or Krusal-Wallis tests. Differences in categorical data can be assessed with Fisher's exact test. In exemplary systems, methods and computer-accessible medium according to exemplary embodiments of the present disclosure, P-values less than 0.05 can be considered statistically significant. The GraphPad Prism software package can be used for all statistical analysis.

Sex as a Biological Variable: The exemplary systems, methods and computer-accessible medium according to the exemplary embodiments of the present disclosure can be utilized for both men and women. In 2023, women comprised 29.4% of cases of catheter ablation for ventricular tachycardia at CUIMC. All results and outcomes can also be summarized by sex, to see if there is an impact of sex on the characterization and identification of the isthmus region.

The exemplary systems, methods and computer-accessible medium according to the exemplary embodiments of the present disclosure can provide fully annotated datasets for all patients enrolled. Although the data processing software contains safeguards to exclude data with an unacceptable signal-to-noise ratio, excessive noise in a large fraction of points can degrade the reliability of the feature extraction. Although ablation of VT is a common procedure in general, mapping of VT circuits is less common due to the hemodynamic instability commonly seen in VT and the demonstrated clinical benefit of substrate ablation compared to ablation with VT circuit mapping. (See, e.g., Ref. 60). For this reason, and because it is the practice at some institutions not to employ empiric percutaneous mechanical support for VT mapping, patients with LVADs can be overrepresented among patients mapped in VT. The exemplary systems, methods and computer-accessible medium according to the exemplary embodiments of the present disclosure can judge the identity of the isthmus based on arrhythmias on presentation (for those that present in arrhythmia) or by recordings of transiently present clinical or induced arrhythmias (for VT patients mapped in a non-VT rhythm), and as a result, it is possible that uninduced arrhythmias utilizing additional isthmuses may not be captured.

### Localizing Reentry Isthmus in Atrial and Ventricular Tachycardia Using Deep Learning

Providing an exemplary predictive model based on EGM analysis can facilitate a real-time guidance of lesion placement to improve success rates and standardize the practice of complex ablation procedures. Within cardiac electrophysiology, there has been substantial work utilizing machine learning methods for analysis of ECGs (see, e.g., Refs. 45, 61-62) and medical images. (See, e.g., Ref. 63, 64). The exemplary systems, methods and computer-accessible medium according to the exemplary embodiments of the present disclosure can contribute towards the foundation of using machine learning for characterizing electroanatomical maps.

### Exemplary Results

The exemplary systems, methods and computer-accessible medium according to the exemplary embodiments of the present disclosure can collect electroanatomic maps from 29 ATs, and calculate/determine custom electrogram features assessing key tissue and wavefront properties for each point. Isthmuses can be labeled off-line. Training data can be used by the exemplary systems, methods and computer-accessible medium according to the exemplary embodiments of the present disclosure to determine the optimal GCN parameters and to train the final model. Putative isthmus points can be predicted in the training and test populations and grouped into proposed isthmus areas based on density and distance thresholds. The exemplary outcome according to exemplary embodiments of the present disclosure can be the shortest distance between the true and proposed isthmus area centroids. A total of 193,821 points can be collected. Thirty isthmuses can be detected in 29 tachycardias among 25 patients (e.g., median age 65.0, 5 women). The median (quartiles) distance between true and the closest proposed isthmus area centroids can be 8.2 (3.5, 14.4) mm in the training and 7.3 (2.8, 16.1) mm in the test group. The mean (+/−standard deviation) overlap in areas, measured by the Dice coefficient, can be 11.5+/−3.2% in the training group and 13.9+/−4.6% in the test group.

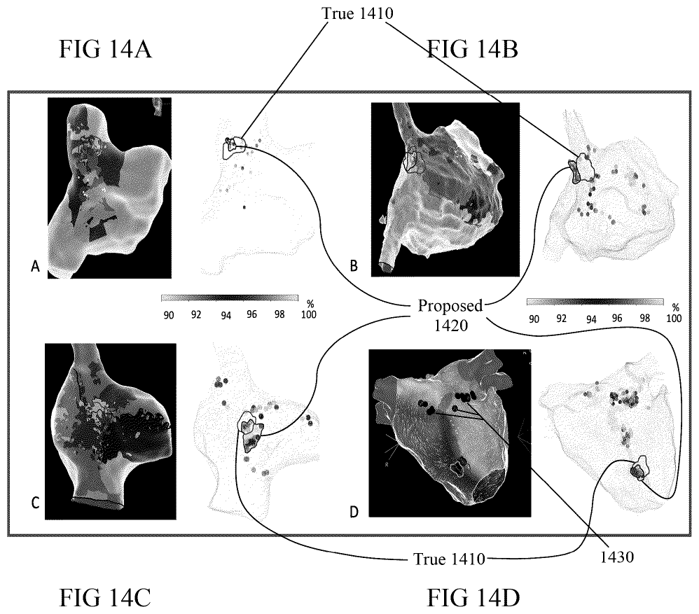

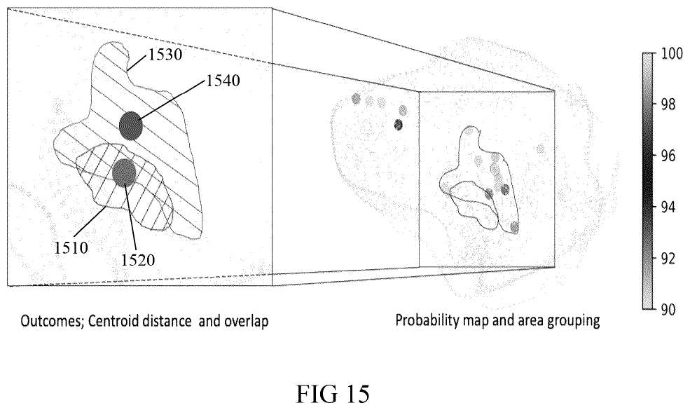

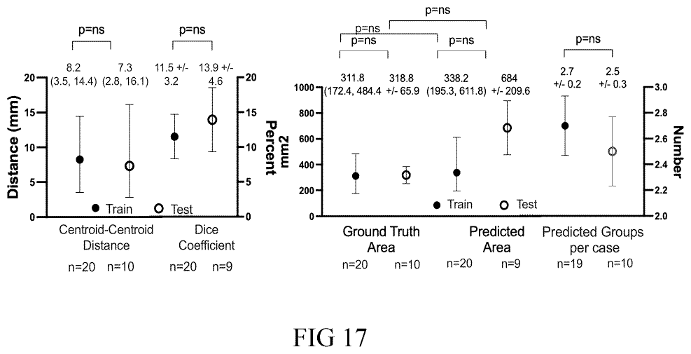

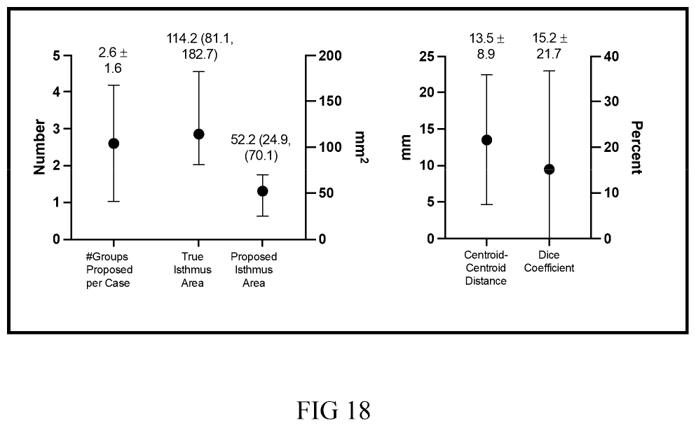

FIGS. 14A-14D show exemplary illustrations of examples of activation and isthmus probability maps derived from maps within AT. Exemplary probability maps are depicted on the right of each figure, and are shown next to activation maps on the left of each figure and illustrate true isthmuses 1410 and closest proposed isthmuses 1420. Black points 1430 are manually placed markers indicating fractionated signals. FIG. 17 illustrates the centroid-centroid and dice coefficient values in training and test groups for atrial tachycardia patients. FIG. 17 also shows bar graphs indicating a comparison of the ground truth and predicted areas, and the number of predicted groups per case for the training and test groups. The exemplary systems, methods and computer-accessible medium according to the exemplary embodiments of the present disclosure can assess feature importance by excluding individual features and observing the subsequent decrease in model performance. In a similarly structured exemplary feasibility study of 10 mapped VTs, isthmuses were predicted to within 13.5+/−8.9 cm with a Dice coefficient of 14.2+/−21.7% (FIGS. 15 and 18).

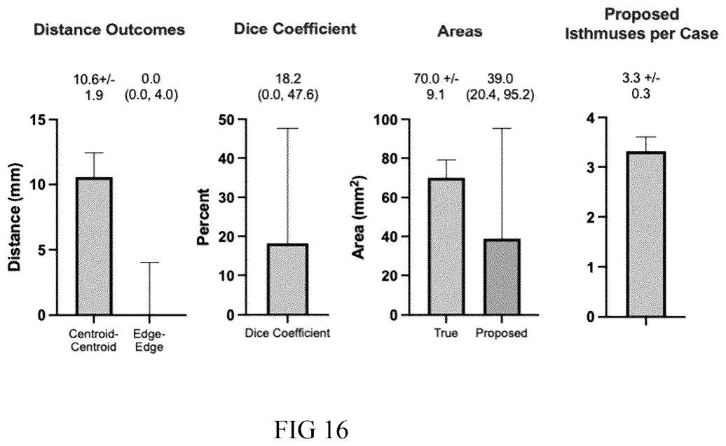

FIG. 15 shows an exemplary illustration of an example of an isthmus probability map in a ventricular tachycardia case. Points with greater than 90% probability are enlarged for clarity. As shown in FIG. 15, the true isthmus 1510 is provided, centroid 1520, and the proposed isthmus 1530 is provided with centroid 1540. FIGS. 16 and 18 illustrate exemplary preliminary results of study outcome metrics for EAM maps of VT. The primary (Centroid-Centroid distance) and secondary outcomes (Dice coefficient). The median true and proposed isthmus areas, and the median number of proposed isthmus areas per case are shown in FIGS. 15, 16 and 18. This exemplary data using the exemplary systems, methods and computer-accessible medium according to the exemplary embodiments of the present disclosure suggest a trained GCN can identify isthmus areas in scar-related tachycardias, and may help identify critical ablation targets.

The exemplary systems, methods and computer-accessible medium according to the exemplary embodiments of the present disclosure can provide fully supervised machine learning methods to identify measurement sites from electroanatomical maps as critical isthmus points. Due to inherent structural and functional differences between the chambers, an initial approach may be to develop separate models for the ventricles and the atria. The exemplary systems, methods and computer-accessible medium according to the exemplary embodiments of the present disclosure can use a supervised learning approach to train machine-learning models to recognize points located on critical isthmuses. First, a logistic regression model can be used to predict if a measurement site belongs to or doesn't belong to the isthmus based on the features derived described herein. Second, a graph-based convolutional neural network (GCN) can be provided that takes the feature vector and the spatial relationship between each measurement site as input.

### Exemplary Data Preconditioning

Providing an automated procedure for identifying the region corresponding to the critical isthmus from electroanatomical mapping is challenging due to data imbalance. Most electrograms acquired within an electroanatomical map are not from the isthmus region. In a review of the dataset from 29 ATs for the exemplary systems, methods and computer-accessible medium according to the exemplary embodiments of the present disclosure, approximately 1.6% of the data points correspond to the isthmus. Therefore, the exemplary systems, methods and computer-accessible medium according to the exemplary embodiments of the present disclosure can employ methods to ensure that the exemplary procedure would not automatically assume that a node is not part of the isthmus due to the imbalance in the dataset. After compiling the entire dataset described herein, if the number of points within the dataset that corresponds to the isthmus remains <5%, the exemplary systems, methods and computer-accessible medium according to the exemplary embodiments of the present disclosure can implement a preprocessing step to downsample the negative samples, making the number of positive points 20% of the dataset for training. In addition, the exemplary systems, methods and computer-accessible medium according to the exemplary embodiments of the present disclosure can apply the focal loss, which assigns special weights to the class that is harder to classify (e.g., usually the class with fewer samples). The exemplary systems, methods and computer-accessible medium according to the exemplary embodiments of the present disclosure can also carry out data (i.e., feature) normalization and augmentation to increase the data for training.

### Exemplary Method 1: Regression

The first exemplary method utilized by the exemplary systems, methods and computer-accessible medium according to the exemplary embodiments of the present disclosure can be the regression method. Here, the input can include the feature vector described herein for each point within the electroanatomical maps. The output can be the probability for each point being part of the critical isthmus. First, according to the exemplary systems, methods and computer-accessible medium according to the exemplary embodiments of the present disclosure, a logistic regression model can be developed. The exemplary systems, methods and computer-accessible medium according to the exemplary embodiments of the present disclosure can use a shallow network with only two hidden layers to avoid overfitting. The exemplary systems, methods and computer-accessible medium according to the exemplary embodiments of the present disclosure can provide hidden layers containing 32 nodes and 16 nodes, and use the rectified linear unit (ReLU) activation function for non-linear feature extraction. The exemplary systems, methods and computer-accessible medium according to the exemplary embodiments of the present disclosure can use focal loss to optimize the network parameters. Further, the exemplary systems, methods and computer-accessible medium according to the exemplary embodiments of the present disclosure can use an ‘Adam’ optimization method with a scheduled learning rate for the gradient descent. This approach may possible not use spatial proximity information.

### Exemplary Method 2: Exemplary Graph Convolutional Neural Network

Traditional convolutional neural networks work on images with regular distance between points. Exemplary electroanatomical maps can be generated by an electrophysiologist navigating a catheter around the chamber, where points are not acquired at fixed or regular spatial sampling intervals. Therefore, the exemplary systems, methods and computer-accessible medium according to the exemplary embodiments of the present disclosure can use a graph convolutional neural network (GCN) (see, e.g., Refs. 65 and 66), instead of a convolutional neural network (CNN) to incorporate spatial relationships between nodes and electrogram features.

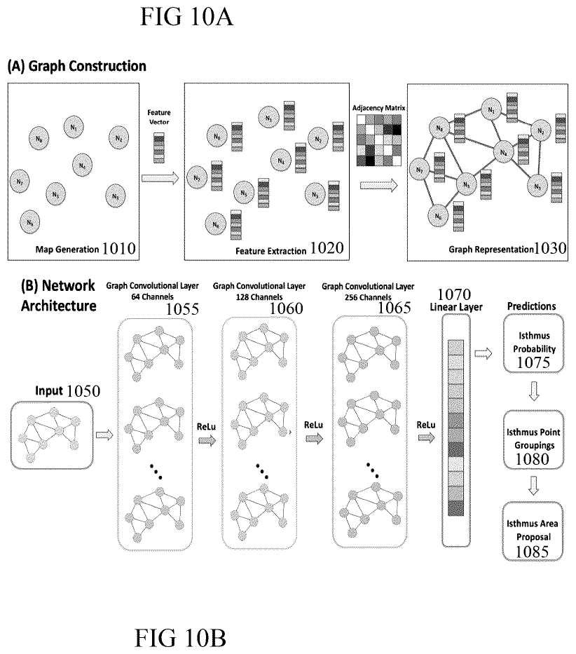

FIGS. 10A and 10B shows a set of exemplary constructed graphs using procedures 1010-1030. First, a map can be generated at step 1010, then features can be extracted at step 1020. Further, a graph representation can be generated at step 1030. Features can be calculated for each point and an adjacency matrix used to define connections. When the graph structure is established, the data can be processed by the network and isthmus area prediction may be made. For example, the input 1050 to the GCN of the exemplary systems, methods and computer-accessible medium according to the exemplary embodiments of the present disclosure can be the feature vector for each node and the adjacency matrix. The adjacency matrix can encode the spatial relationships (“edges”) between nodes. This can occur through graphing convolutional layers in 64 channels at step 1055, then at 128 channels at step 1060, then at 256 channels at step 1065. The exemplary systems, methods and computer-accessible medium according to the exemplary embodiments of the present disclosure can use a weighted graph, whereas the entries of the adjacency matrix are the geodesic distance between nodes. A linear layer can be generated at step 1070, leading to the ability to make predictions. For example, isthmus probability may be predicted at step 1075, isthmus point groupings may be predicted at step 1080, and isthmus area proposals may be provided at step 1085.

The exemplary systems, methods and computer-accessible medium according to the exemplary embodiments of the present disclosure can make predictions of each node, such as node classification and link prediction. The exemplary systems, methods and computer-accessible medium according to the exemplary embodiments of the present disclosure can use this computation graph to propagate aggregated information from neighbors to make a prediction. This propagation can capture the network structure and the information from nearby neighbors. A variable in this analysis can include the maximum separation distance for two measurement sites to be considered neighbors. For example, if a measurement site is in an infarct area of low voltage, there can be a higher probability that neighboring nodes may be part of the critical isthmus region. The exemplary systems, methods and computer-accessible medium according to the exemplary embodiments of the present disclosure can test different threshold values for distance, which can be used to update the adjacency matrix. The exemplary systems, methods and computer-accessible medium according to the exemplary embodiments of the present disclosure can begin with a GCN model with two hidden layers (e.g., 32 and 64 neurons) using a ReLU activation function. The network can be optimized by the cross-entropy loss with a stepped learning rate. The model output, generated using the exemplary systems, methods and computer-accessible medium according to the exemplary embodiments of the present disclosure, can be a probability and classification for each node being part or not part of the critical isthmus.

The exemplary system, method and computer-accessible medium according to the exemplary embodiments of the present disclosure can be implemented, for example, on a computer with the following specifications for the GPU and CPU: Intel core i9-9900K (16M Cache, up to 5.00 GHz) CPU and an RTX2080 GPU, respectively. Exemplary systems, methods and computer-accessible medium according to exemplary embodiments of the present disclosure can use PyTorch for the deep learning framework.

Exemplary Model Output: In exemplary systems, methods and computer-accessible medium according to exemplary embodiments of the present disclosure, for every point presented to the DLN, the probability of that point being an isthmus point can calculated. Training iteratively modifies this calculation until the performance of the DLN compared to the ground truth is optimized. In all experiments, points with an isthmus probability of, e.g., >90%, can be considered to be predicted isthmus points.

### Exemplary Method 3: PointNet and PointNet++

PointNet is a deep learning analysis procedure (see, e.g., Ref. 67) that uses feature vectors from unordered point clouds such as those generated through electroanatomic mapping to perform both classification and segmentation. The further benefits include (1) invariance to data feeding order, (2) leveraging of global and local point features, and (3) invariance to rotational and translational transformations. Its three core modules include a transformation network (T-net), a symmetric function (max-pooling), and the multi-layer perceptron. A limitation of PointNet is its limited ability to detect intermediate hierarchical structures. (See, e.g., Ref. 67). PointNet++uses an iterative farthest point sampling algorithm (FPS) to identify points that equally span the whole point cloud (see, e.g., Ref. 68), better classifying geometric relationships of local point groups.

Both exemplary procedures have been used extensively in studying point cloud structure in the life sciences. (See, e.g., Refs. 69 and 70). The exemplary systems, methods and computer-accessible medium according to the exemplary embodiments of the present disclosure can use these structures as initial architectural frameworks for automatic segmentation of electroanatomic point clouds into isthmus regions using the optimized features determined as input feature vectors.

### Exemplary Method 4: Multi-Channel Image Classification Using Convolutional Network (CNN)

Standard 2D image classification uses a CNN on 3-channeled regularly-spaced data. For each arrhythmia mapped using the exemplary systems, methods and computer-accessible medium according to the exemplary embodiments of the present disclosure, a regularly-ordered image can be generated by interpolation. Associated multi-channel features extracted as described herein can then be applied to a standard CNN, generating a probability distribution for each point in the image map. Standard image processing augmentation methods (flipping, adding Gaussian noise to the features, etc.) can be employed within the training set to further generalize the network.

### Exemplary Outcomes and Evaluation Metrics

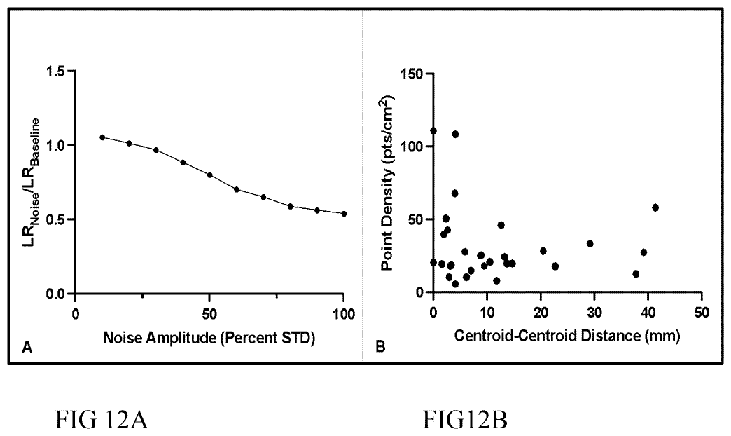

The primary outcome using the exemplary systems, methods and computer-accessible medium according to the exemplary embodiments of the present disclosure can be the distance between the centroids of the true and the closest proposed isthmus area (see FIGS. 17 and 18). The true isthmus can be defined by offline analysis as described within the shared methods section above. The exemplary systems, methods and computer-accessible medium according to the exemplary embodiments of the present disclosure can yield a distance between centroids of 10.6 (1.9) mm. The exemplary systems, methods and computer-accessible medium according to the exemplary embodiments of the present disclosure can optimize the network to have a distance between the centroids of less than 6 mm, the average diameter of an ablation lesion. (See, e.g., Ref. 71). The secondary outcome with the use of the exemplary systems, methods and computer-accessible medium according to the exemplary embodiments of the present disclosure can be the amount of overlap between these areas as measured by the Dice coefficient (see FIGS. 11A-11D and 12A-12B). When more than one true isthmus is identified, outcomes can be calculated separately for each.

Exemplary Effects of data noise and point density on model performance: To assess the robustness of the exemplary model according to to the exemplary embodiments of the present disclosure, to variations in data quality, randomly generated scaled noise can be added to the test data at increasing intensity, and the ratio between the resulting and baseline LRs can be calculated or otherwise determined. For example, the model's likelihood ratio can decrease to approximately 80% of baseline when the standard deviation of the introduced noise is 50% that of the feature value. Using the exemplary systems, methods and computer-accessible medium according to the exemplary embodiments of the present disclosure, there can be a nonsignificant trend towards improved centroid-centroid distance with increased point density. In addition, exemplary systems, methods and computer-accessible medium according to exemplary embodiments of the present disclosure can evaluate the effect of EAM point density on the primary outcome of centroid-centroid distance, as shown in FIG. 12. FIGS. 12A and 12B also show the graphs indicating the robustness of a model, by using the exemplary systems, methods and computer-accessible medium according to the exemplary embodiments of the present disclosure, to introduced noise as measured by the ratio of the likelihood ratios (LR) when the noise is introduced compared to baseline LR with different levels if introduced noise Target outcomes can be the distance being the centroid of the proposed isthmus and to the centroid of the true isthmus of less than 6 mm, approximately the diameter of an ablation lesion.

It is possible that—although the average point density can be sufficient to characterize the isthmus location—that there can be insufficient regional point density to include a reasonable number of points on the isthmus. In some exemplary cases, there may be ambiguity in the exact borders of the isthmus. The exemplary systems, methods and computer-accessible medium according to the exemplary embodiments of the present disclosure can provide graph-based convolutional neural networks that can perform better than the logistic regression model. While the dataset for the logistic regression is larger, as each sample measurement site can be considered a datapoint, the GCN can include spatial relationships through the adjacency matrix. A decreased centroid-to-centroid distance and increased DICE coefficient can be achieved by including additional post processing steps to reduce the classification map complexity by removing isolated points identified as belonging to the isthmus and/or using ensemble networks and voting. Some challenges are that the density of the maps will vary by patient. Since the overwhelming majority of the maps are expected to be acquired endocardially, any epicardial maps will not be included in the training set.

The exemplary systems, methods and computer-accessible medium according to the exemplary embodiments of the present disclosure establish a new quantitative paradigm in electroanatomical analysis of scar-mediated reentry. The exemplary procedures provided in connection with the exemplary systems, methods and computer-accessible medium according to the exemplary embodiments of the present disclosure can facilitate operators to target critical areas in a manner more targeted than the current standard of care, ultimately reducing procedure time and potentially improving ablation success rates. In so doing, the exemplary ablation procedures can be more targeted, minimizing procedure time and maximizing procedural success.

The exemplary software can be written in the Python script, and can utilize files generated by commercial electroanatomic mapping systems created using Biosense Webster (CARTO) or Abbott (EnSite Precision). The exemplary software can receive the signals recorded at each point and analyze the signal for various features (see examples in Table 2 below).

TABLE 2

| Feature | Explanation |
|---|---|
| Power | The power of the signal in dB |
| Percent Signal | The percentage of time a signal is above |
| | or below a threshold value |
| Amplitude | The amplitude of the signal |
| Peak Frequency | The peak frequency of the signal as |
| | determined by the Fourier transform |
| Signal Width | The width of the electrogram |
| X, Y, Z | X, Y, and Z coordinates of the measured point |
| CV | Conduction velocity |
| CV X, Y, Z | The vector of electrical propagation at each point |
| Coherence | The percentage of vectors in a region that differ |
| | from the index vector by greater than a |
| | threshold angle |
| Mean Angle | The average of the angle a region's vectors |
| | make with the index vector |
| Block | Points whose activation suggest they are |
| | sites of conduction block |
| Largest Regional | The largest difference in activation time |
| activation difference | between the index point and regional points |

The exemplary features for the characterization of isthmuses can include, e.g., voltage, conduction velocity and direction, and electrogram width, as discussed herein. Depending on the system used, bipolar voltage amplitude is either exported directly or can be calculated as positive-to-negative electrogram height. Conduction velocity and vector direction can be derived or otherwise obtained by trigonometric triangulation. (See, e.g., Ref. 16). Exemplary calculations of the signal's instantaneous energy (see, e.g., Ref. 17) facilitates the calculation of electrogram width and the detection of double potentials (see, e.g., FIG. 3). Applying a radius around each sampled point to define an individual point's region, summary statistics for regional vector coherence and the average angle between conduction vectors can be calculated. In a similar way, by defining a lower physiologic limit of conduction velocity, regions of conduction block can be identified.

The exemplary information about the features at each point, the distance between the points, and the geometry of the chamber studied is summarized in a series of files. This information can then be applied to a deep learning network to train the network to recognize isthmuses.

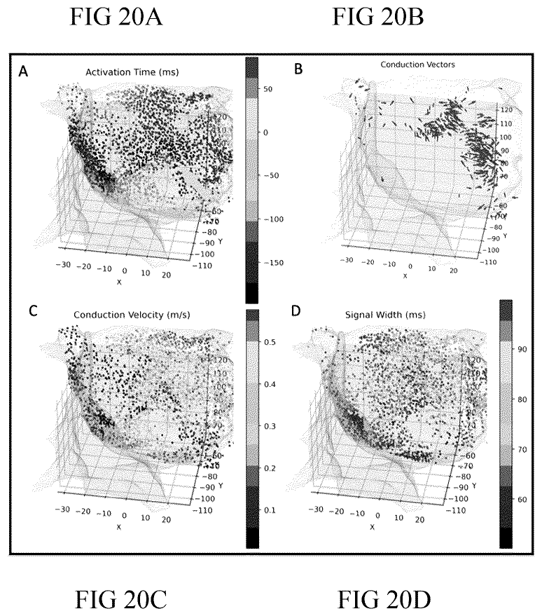

FIGS. 20A-20D show exemplary outputs of the exemplary software from a test case, e.g., of an atrial tachycardia utilizing a critical region of scarring on the anterior wall of a left atrium (arrow, FIG. 20A) with conduction vectors displayed for about 20 ms of activation time (FIG. 20B), conduction velocity (FIG. 20C), and signal width (FIG. 20D).

### Exemplary Methods

Patients undergoing VT ablation during tachycardia using the Abbott or CARTO systems were screened. Thirteen features assessing EGM morphology and wavefront propagation were calculated for each point. Known isthmuses were labelled. A leave-one-out cross-validation strategy was used, wherein each case was successively sequestered as a test case, while the remaining were used for training. During testing, an isthmus probability map was generated, and points with a >90% probability were grouped into proposed isthmus areas based on density and distance thresholds. The primary outcome was distance between the centroids of the closest proposed and the true isthmus areas.

In particular, successful ablations of a scar-related VT mapped during arrhythmia were performed. Isthmuses were identified by consensus opinion (DS and EC). Features were calculated for each point using software (e.g., Python 3.8). FIGS. 2 and 3 show a set of exemplary graphs providing exemplary calculation(s) of EGM width, number of potentials, and potential spacing using a calculation of instantaneous energy, according to exemplary embodiments of the present disclosure. The exemplary results are provided in Table 3 below:

TABLE 3
Exemplary List of features used for network training.

| Feature | Description | Feature | Description |
|---|---|---|---|
| Minimal | Minimal ΔLAT/distance between a point | Signal Power | EGM signal power (dB). |
| Regional | and its regional neighbors | | |
| Conduction | | | |
| Speed (MRCS) | | | |
| Distance to | Distance between a point and its closest | Signal | The percentage of the EGM signal |
| scar | neighbor with voltage consistent with | Percentage | above the noise threshold. |
| | scar. | | |
| Distance to | Distance between a point and its closest | Number of | Number of complexes detected in an |
| block | neighbor with MRCS consistent with | Deflections^{†} | EGM |
| | block. | | |
| Distance to | Distance between a point and its closest | Percent Vector | Percentage of neighboring vectors with |
| scar or block | neighbor with MRCS consistent with | Incoherencet | an angle of incidence >45°. |
| | block and/or voltage consistent with scar. | | |
| Distance to | Shortest distance between a point and a | Forward | Maximum difference in LAT between a |
| barrier^{†} | valve annulus, conduction block or scar. | Maximal LAT | point and the subsequently activated |
| | | Difference | points within a 5 mm radius. |
| | | (FMLATD) | |
| Double | Maximal duration of baseline between | Peak Frequency | Peak frequency Fourier-transformed |
| potential | complexes for multicomponent EGMs. | | EGM signal measured up to 12 Hz |
| spacing | | | |
| Conduction | Wavefront activation vector | Maximum | Maximum width of any deflection in the |
| Velocity (CV) | | Deflection | EGM |
| | | Width^{a} | |
| Voltage^{‡} | Peak-to-peak EGM amplitude | Width of | Width of the deflection with the |
| | | Maximal | maximum amplitude in the EGM |
| | | Amplitude | |
| | | Deflection^{†} | |
| Mean Vector | The angle between a point's conduction | Total Signal | Sum of the widths of all deflections |
| Angle | vector and the mean vector | Width^{†} | recorded in the EGM |
| | of its regional neighbors | | |
| LAT Range | Maximal ΔLATs between a | | |
| | point and its regional neighbors | | |

By marking ground-truth isthmus points, a supervised training of a GCN was performed, as shown in FIGS. 10A and 10B, which illustrates flow diagrams and structure of an exemplary graph convolutional network according to an exemplary embodiment of the present disclosure. In an exemplary leave-one-out validation analysis, exemplary isthmus probability maps were created for each case. Adjacent points with great than about 90% probability were grouped into proposed isthmus areas.

The primary exemplary outcome was the geodesic distance between the closest predicted and the true isthmus area centroids. The secondary exemplary outcome was the area overlap measured by the Dice Coefficient. For example, FIG. 15 shows an exemplary illustration of a grouping of high-probability isthmus points into areas and visualization of the primary and secondary outcomes according to an exemplary embodiment of the present disclosure.

### Exemplary Results

Eleven LV tachycardias were mapped in 10 patients (median age 69, 100% male), 7 with ischemic VT and 3 non-ischemic. Two patients had an LVAD. One VT (9.1%) was epicardial. Exemplary results are provided below in Table 4. Ablations were acutely successful in all patients.

TABLE 4
Exemplary characteristics of included VTs

| | Map | | | | Ischemic/ | | Epi/ | | |
|---|---|---|---|---|---|---|---|---|---|
| | Type | Age | EF | M/F | Nonischemic | LVAD | endocardial | Antiarrhythmics | Location |
| 1 | CARTO | 79 | 35 | M | NI | N | Endo | Amiodarone | Anteroseptal |
| 2 | Abbott | 73 | 15 | M | I | N | Endo | Sotalol | Posterolateral |
| 3 | CARTO | 64 | 45 | M | NI | N | Epi | Amiodarone | Inferior |
| 4 | Abbott | 74 | 15 | M | I | Y | Endo | | Anteroseptal |
| 5 | Abbott | 53 | 35 | M | I | N | Endo | Sotalol | Posterolateral |
| 6 | Abbott | 65 | 15 | M | I | N | Endo | None | Apical lateral |
| 7 | Abbott | 64 | 15 | M | NI | N | Endo | None | Anteroseptal |
| 8 | Abbott | 83 | 25 | M | I | N | Endo | Amiodarone | Anteroseptal |
| 9 | Abbott | 65 | 15 | M | I | N | Endo | Amiodarone | Posterolateral |
| 10 | Abbott | 76 | 30 | M | I | Y | Endo | Sotalol, mexilitine | Inferolateral |
| 11 | Abbott* | — | — | | — | — | Endo | Sotalol, mexilitine | Inferolateral |
| Median | 69 | 20 | *VT10 and 11 were induced in the same patient | | | | | | |
| (IQR) | (64, 76) | (15, 35) | | | | | | | |

A total of 8033 points were analyzed. Of these, 491 (6.1%) were grouped into proposed isthmus areas. No prediction was made in one case. Among cases with predictions, there was 2.6 +/−0.5 proposed isthmus areas per case. The distance between centroids was 13.5+/−2.8 mm. The mean area overlap, measured by the Dice coefficient, was 15.2+/−6.8%. Indeed, FIG. 18 shows an exemplary illustration of primary and secondary outcomes according to an exemplary embodiment of the present disclosure.

### Exemplary Conclusions

Exemplary EGM features can be calculated and used to train a GCN that can predict isthmus areas in VT and may help identify critical sites for ablation in their treatment. Indeed, the trained GCN can be used in predicting isthmus areas in scar-related VT with reasonable accuracy. Such trained GNC can be used for atrial arrhythmias, chamber-specific arrhythmias, or data collected in sinus rhythms.

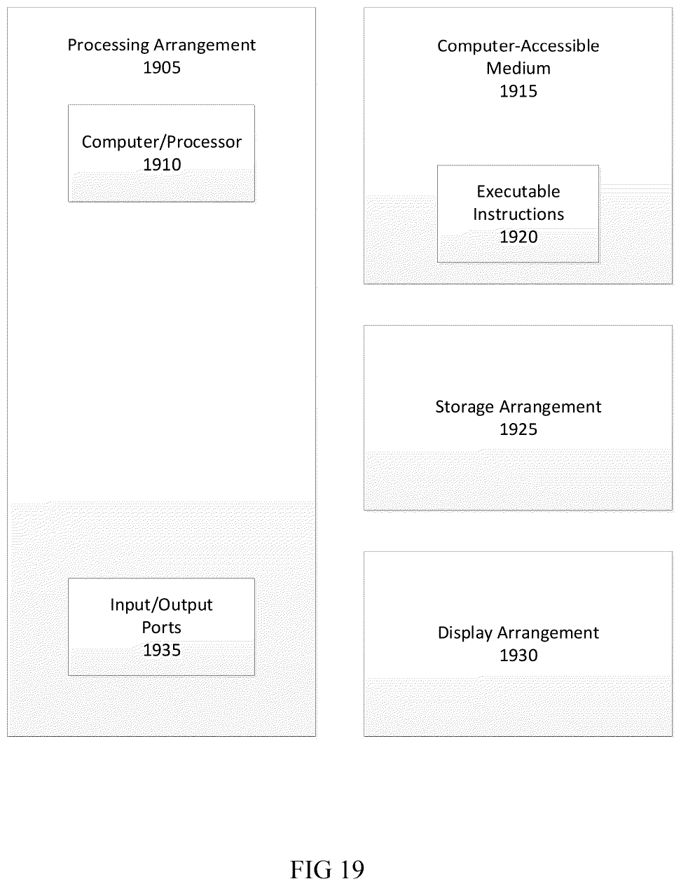

FIG. 19 shows a block diagram of an exemplary embodiment of a system according to the present disclosure, which can be utilized either in part or completely with any one or more of the exemplary embodiments of the present disclosure as provided in the enclosed Appendix. For example, exemplary procedures in accordance with the present disclosure described herein can be performed by a processing arrangement and/or a computing arrangement 1902. Such processing/computing arrangement 1902 can be, for example entirely or a part of, or include, but not limited to, a computer/processor 1904 that can include, for example one or more microprocessors, and use instructions stored on a computer-accessible medium (e.g., RAM, ROM, hard drive, or other storage device).

As shown in FIG. 19, for example a computer-accessible medium 1906 (e.g., as described herein above, a storage device such as a hard disk, floppy disk, memory stick, CD-ROM, RAM, ROM, etc., or a collection thereof) can be provided (e.g., in communication with the processing arrangement 1902). The computer-accessible medium 1906 can contain executable instructions 1908 thereon. In addition, or alternatively, a storage arrangement 1910 can be provided separately from the computer-accessible medium 1906, which can provide the instructions to the processing arrangement 1902 so as to configure the processing arrangement to execute certain exemplary procedures, processes and methods, as described herein above, for example.

Further, the exemplary processing arrangement 1902 can be provided with or include an input/output arrangement 1914, which can include, for example a wired network, a wireless network, the internet, an intranet, a data collection probe, a sensor, etc. FIG. 19 shows that the exemplary processing arrangement 1902 can be in communication with an exemplary display arrangement 1912, which, according to certain exemplary embodiments of the present disclosure, can be a touch-screen configured for inputting information to the processing arrangement in addition to outputting information from the processing arrangement, for example. Further, the exemplary display 1912 and/or a storage arrangement 1910 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are hereby incorporated by reference in their entireties.
1. Waldo, A. L., Plumb, V. J., Arciniegas, J. G., MacLean, W. A., Cooper, T. B., Priest, M. F., & James, T. N. (1983). Transient entrainment and interruption of the atrioventricular bypass pathway type of paroxysmal atrial tachycardia. a model for understanding and identifying reentrant arrhythmias. Circulation, 67 (1), 73-83. https://doi.org/10.1161/01.cir.67.1.73.
2. Aziz, Z., Shatz, D., Raiman, M., Upadhyay, G. A., Beaser, A. D., Besser, S. A., Shatz, N. A., Fu, Z., Jiang, R., Nishimura, T., Liao, H., Nayak, H. M., & Tung, R. (2019). Targeted ablation of ventricular tachycardia guided by wavefront discontinuities during sinus rhythm. Circulation, 140 (17), 1383-1397. https://doi.org/10.1161/circulationaha.119.042423.
3. Martin, R., P. Maury, C. Bisceglia, T. Wong, H. Estner, C. Meyer, C. Dallet, C. Martin, R. Shi, et al., Characteristics of scar-related ventricular tachycardia circuits using ultra-high-density mapping: a multi-center study. Circulation: Arrhythmia and Electrophysiology, 2018. 11 (10): p. e006569.
4. Tschabrunn, C., S. Roujol, R. Nezafat, B. Faulkner-Jones, A. Buxton, M. Josephson, and E. Anter, A swine model of infarct-related reentrant ventricular tachycardia: electroanatomic, magnetic resonance, and histopathological characterization. Heart rhythm, 2016. 13 (1): p. 262-73.
5. Lin, C., J. Silberbauer, Y. Lin, M. Lo, C. Lin, H. Chang, S. Chang, L. Lo, Y. Hu, et al., Simultaneous amplitude frequency electrogram transformation (SAFE-T) mapping to identify ventricular tachycardia arrhythmogenic potentials in sinus rhythm. JACC: Clinical Electrophysiology, 2016. 2 (4): p. 459-70.
6. Martin, R., P. Maury, C. Bisceglia, T. Wong, H. Estner, C. Meyer, C. Dallet, C. Martin, R. Shi, et al., Characteristics of scar-related ventricular tachycardia circuits using ultra-high-density mapping: a multi-center study. Circulation: Arrhythmia and Electrophysiology, 2018. 11 (10): p. e006569.
7. Frontera, A., S. Pagani, L. Limite, A. Hadjis, A. Manzoni, L. Dede, A. Quarteroni, and P. Della Bella, The Outer Loop and Isthmus in Ventricular Tachycardia Circuits: Characteristics and Implications. Heart Rhythm, 2020. 17 (10).
8. Ciaccio, E. J., Premature excitation and onset of reentrant ventricular tachycardia. Am J Physiol Heart Circ Physiol, 2002. 283 (4): p. H1703-12
9. Ciaccio, E., A. Tosti, and M. Scheinman, Relationship between sinus rhythm activation and the reentrant ventricular tachycardia isthmus. Circulation, 2001. 104 (5): p. 613-9.
10. Irie, T., R. Yu, J. Bradfield, M. Vaseghi, E. Buch, O. Ajijola, C. Macias, O. Fujimura, R. Mandapati, et al., Relationship between sinus rhythm late activation zones and critical sites for scar-related ventricular tachycardia: systematic analysis of isochronal late activation mapping. Circ Arrhythm Electrophysiol, 2015. 8: p. 390-399.
11. Aziz, Z., D. Shatz, M. Raiman, G. Upadhyay, A. Beaser, S. Besser, N. Shatz, Z. Fu, R. Jiang, et al., Targeted Ablation of Ventricular Tachycardia Guided by Wavefront Discontinuities During Sinus Rhythm: A New Functional Substrate Mapping Strategy. Circulation, 2019. 140 (17): p. 1383-1397.
12. Kalman, J., G. VanHare, J. Olgin, L. Saxon, S. Stark, and M. Lesh, Ablation of ‘incisional’ reentrant atrial tachycardia complicating surgery for congenital heart disease. Use of entrainment to define a critical isthmus of conduction. Circulation, 1996. 93 (3): p. 502-512
13. Chae, S., H. Oral, E. Good, S. Dey, A. Wimmer, T. Crawford, D. Wells, J.-F. Sarrazin, N. Chalfoun, et al., Atrial tachycardia after circumferential pulmonary vein ablation of atrial fibrillation: mechanistic insights, results of catheter ablation, and risk factors for recurrence. J Am Coll Cardiol. 2007. 50 (18): p. 1781-1787.
14. Latcu, D., S. Bun, F. Viera, T. Delassi, M. El Jamili, A. Al Amoura, and N. Saoudi, Selection of Critical Isthmus in Scar-Related Atrial Tachycardia Using a New Automated Ultrahigh Resolution Mapping System. Circ Arrhythm Electrophysiol., 2017. 10 (1): p. e004510.
15. Oesterlein, Tobias, Kramlich, Alexander, Lenis, Gustavo, Luik, Armin, Schmitt, et al., Automatic detection and mapping of double potentials in intracardiac electrograms. Current Directions in Biomedical Engineering. 2 (1): p. 179-183.
16. Cantwell, C., C. Roney, F. Ng, J. Siggers, S. Sherwin, and N. Peters, Techniques for automated local activation time annotation and conduction velocity estimation in cardiac mapping. Comput Biol Med, 2015. 65: p. 229-42.
17. O'Toole, J., A. Temko, and N. Stevenson, Assessing instantaneous energy in the EEG: a non-negative, frequency-weighted energy operator. Annu Int Conf IEEE Eng Med Biol Soc, 2014: p. 3288-91.
18. Di Diego J M, Antzelevitch C. Ischemic ventricular arrhythmias: experimental models and their clinical relevance. Heart Rhythm. 2011 December; 8 (12): 1963-1968. PMCID: PMC3222739
19. Wasmer K, Monnig G, Bittner A, Dechering D, Zellerhoff S, Milberg P, Kobe J, Eckardt L. Incidence, characteristics, and outcome of left atrial tachycardias after circumferential antral ablation of atrial fibrillation. Heart Rhythm. 2012 October; 9 (10): 1660-1666. PMID: 22683745
20. Quinto L, Sanchez-Somonte P, Alarcón F, Garre P, Castillo À, Antonio R S, Borras R, Guasch E, Arbelo E, Tolosana J M, Berruezo A, Mont L, Roca-Luque I. Ventricular tachycardia burden reduction after substrate ablation: Predictors of recurrence. Heart Rhythm. Elsevier; 2021 Jun. 1; 18 (6): 896-904. PMID: 33639298
21. Marchlinski F E, Haffajee C I, Beshai J F, Dickfeld T M L, Gonzalez M D, Hsia H H, Schuger C D, Beckman K J, Bogun F M, Pollak S J, Bhandari A K. Long-Term Success of Irrigated Radiofrequency Catheter Ablation of Sustained Ventricular Tachycardia: Post-Approval THERMOCOOL V T Trial. J Am Coll Cardiol. 2016 Feb. 16; 67 (6): 674-683. PMID: 26868693
22. Cronin E M, Bogun F M, Maury P, Peichl P, Chen M, Namboodiri N, Aguinaga L, Leite L R, Al-Khatib S M, Anter E, Berruezo A, Callans D J, Chung M K, Cuculich P, d'Avila A, Deal B J, Bella P D, Deneke T, Dickfeld T M, Hadid C, Haqqani H M, Kay G N, Latchamsetty R, Marchlinski F, Miller J M, Nogami A, Patel A R, Pathak R K, Saenz Morales L C, Santangeli P, Sapp J L Jr, Sarkozy A, Soejima K, Stevenson W G, Tedrow U B, Tzou W S, Varma N, Zeppenfeld K. 2019 HRS/EHRA/APHRS/LAHRS expert consensus statement on catheter ablation of ventricular arrhythmias. J Interv Card Electrophysiol. 2020 October; 59 (1): 145-298. PMCID: PMC7223859
23. Maagh P, Christoph A, Dopp H, Mueller M S, Plehn G, Meissner A. High-Density Mapping in Ventricular Tachycardia Ablation: A PentaRay® Study. Cardiol Res Pract. 2017 December; 8 (6): 293-303. PMCID: PMC5755661
24. Gandhavadi M, Cox E J. Prevalence of intra-atrial reentrant tachycardia following mitral valve surgery: Relationship to surgical approach. J Card Surg. 2020 August; 35 (8): 1871-1876. PMID:
25. Chugh A, Oral H, Lemola K, Hall B, Cheung P, Good E, Tamirisa K, Han J, Bogun F, Pelosi F Jr, Morady F. Prevalence, mechanisms, and clinical significance of macroreentrant atrial tachycardia during and following left atrial ablation for atrial fibrillation. Heart Rhythm. 2005 May; 2 (5): 464-471. PMID: 15840468
26. Nerheim P, Birger-Botkin S, Piracha L, Olshansky B. Heart failure and sudden death in patients with tachycardia-induced cardiomyopathy and recurrent tachycardia. Circulation. 2004 Jul. 20; 110 (3): 247-252. PMID: 15226218
27. Martin C A, Takigawa M, Martin R, Maury P, Meyer C, Wong T, Shi R, Gajendragadkar P, Frontera A, Cheniti G, Thompson N, Kitamura T, Vlachos K, Wolf M, Bourier F, Lam A, Duchâteau J, Massoullie G, Pambrun T, Denis A, Derval N, Hocini M, Haïssaguerre M, Jaïs P, Sacher F. Use of Novel Electrogram “Lumipoint” Algorithm to Detect Critical Isthmus and Abnormal Potentials for Ablation in Ventricular Tachycardia. JACC Clin Electrophysiol. 2019 April; 5 (4): 470-479. PMID: 31000101
28. Liu S H, Lin Y J, Lee P T, Vicera J J, Chang S L, Lo L W, Hu Y F, Chung F P, Tuan T C, Chao T F, Liao J N, Chang T Y, Lin C Y, Wu C I, Liu C M, Cheng W H, Chen S A. The isthmus characteristics of scar-related macroreentrant atrial tachycardia in patients with and without cardiac surgery. J Cardiovasc Electrophysiol. 2021 July; 32 (7): 1921-1930. PMID: 33834555
29. Vicera JJB, Lin Y J, Lee P T, Chang S L, Lo L W, Hu Y F, Chung F P, Lin C Y, Chang T Y, Tuan T C, Chao T F, Liao J N, Wu C I, Liu C M, Lin C H, Chuang C M, Chen C C, Chin C G, Liu S H,
Cheng W H, Tai L P, Huang S H, Chou C Y, Lugtu I, Liu C H, Chen S A. Identification of critical isthmus using coherent mapping in patients with scar-related atrial tachycardia. J Cardiovasc Electrophysiol. 2020 June; 31 (6): 1436-1447. PMCID: PMC7383970
30. Van Nieuwenhuyse E, Martinez-Mateu L, Saiz J, Panfilov A V, Vandersickel N. Directed graph mapping exceeds phase mapping in discriminating true and false rotors detected with a basket catheter in a complex in-silico excitation pattern. Comput Biol Med. 2021 June; 133:104381. PMCID: PMC8204274
31. Tung R, Vaseghi M, Frankel D S, Vergara P, Di Biase L, Nagashima K, Yu R, Vangala S, Tseng C H, Choi E K, Khurshid S, Patel M, Mathuria N, Nakahara S, Tzou W S, Sauer W H, Vakil K, Tedrow U, Burkhardt J D, Tholakanahalli V N, Saliaris A, Dickfeld T, Weiss J P, Bunch T J, Reddy M, Kanmanthareddy A, Callans D J, Lakkireddy D, Natale A, Marchlinski F, Stevenson W G, Della Bella P, Shivkumar K. Freedom from recurrent ventricular tachycardia after catheter ablation is associated with improved survival in patients with structural heart disease: An International VT Ablation Center Collaborative Group study. Heart Rhythm. 2015 September; 12 (9): 1997-2007. PMCID: PMC4549209
32. Hawson J, Al-Kaisey A, Anderson R D, Watts T, Morton J, Kumar S, Kistler P, Kalman J, Lee G. Substrate-based approaches in ventricular tachycardia ablation. Indian Pacing Electrophysiol J. 2022 Aug. 23; 22 (6): 273-285. PMCID: PMC9649336
33. Klein L S, Shih H T, Hackett F K, Zipes D P, Miles W M. Radiofrequency catheter ablation of ventricular tachycardia in patients without structural heart disease. Circulation. 1992 May; 85 (5): 1666-1674. PMID: 1572025
34. Hsia H H, Lin D, Sauer W H, Callans D J, Marchlinski F E. Anatomic characterization of endocardial substrate for hemodynamically stable reentrant ventricular tachycardia: identification of endocardial conducting channels. Heart Rhythm. 2006 May; 3 (5): 503-512. PMID: 16648052
35. Josephson M E, Anter E. Substrate Mapping for Ventricular Tachycardia: Assumptions and Misconceptions. JACC Clin Electrophysiol. 2015 October; 1 (5): 341-352. PMID: 29759461
36. Ciaccio E J, Coromilas J, Wan E Y, Yarmohammadi H, Saluja D S, Biviano A B, Wit A L, Peters N S, Garan H. Slow uniform electrical activation during sinus rhythm is an indicator of reentrant V T isthmus location and orientation in an experimental model of myocardial infarction. Comput Methods Programs Biomed. 2020 November; 196:105666. PMID: 32717622
37. Ciaccio E J, Coromilas J, Costeas C A, Wit A L. Sinus rhythm electrogram shape measurements are predictive of the origins and characteristics of multiple reentrant ventricular tachycardia morphologies. J Cardiovasc Electrophysiol. 2004 November; 15 (11): 1293-1301. PMID: 15574181
38. Martin R, Hocini M, Haïsaguerre M, Jaïs P, Sacher F. Ventricular Tachycardia Isthmus Characteristics: Insights from High-density Mapping. Arrhythm Electrophysiol Rev. 2019 March; 8 (1): 54-59. PMCID: PMC6434507
39. Acharya U R, Fujita H, Lih O S, Hagiwara Y, Tan J H, Adam M. Automated detection of arrhythmias using different intervals of tachycardia ECG segments with convolutional neural network. Inf Sci. 2017 Sep. 1; 405:81-90.
40. Hannun A Y, Rajpurkar P, Haghpanahi M, Tison G H, Bourn C, Turakhia M P, Ng A Y. Cardiologist-level arrhythmia detection and classification in ambulatory electrocardiograms using a deep neural network. Nat Med. 2019 January; 25 (1): 65-69. PMCID: PMC6784839
41. Zou F, Brahier M, Migliarese F, Ma X, Wu C, Taylor A, Sandfort V, Thomaides A, Vargas J. B-PO03-076 A MACHINE LEARNING-DERIVED RECURRENCE RISK MODEL FOR ATRIAL FIBRILLATION AFTER CATHETER ABLATION. Heart Rhythm. Elsevier; 2021 Aug. 1; 18 (8): S219-S220.
42. Bieging E T, Morris A, Chang L, Dagher L, Marrouche N F, Cates J. Statistical shape analysis of the left atrial appendage predicts stroke in atrial fibrillation. Int J Cardiovasc Imaging. 2021 August; 37 (8): 2521-2527. PMCID: PMC8316337
43. Seitz J, Durdez T M, Albenque J P, Pisapia A, Gitenay E, Durand C, Monteau J, Moubarak G, Theodore G, Lepillier A, Zhao A, Bremondy M, Maluski A, Cauchemez B, Combes S, Guyomar Y, Heuls S, Thomas O, Penaranda G, Siame S, Appetiti A, Milpied P, Bars C, Kalifa J. Artificial intelligence software standardizes electrogram-based ablation outcome for persistent atrial fibrillation. J Cardiovasc Electrophysiol. Wiley; 2022 November; 33 (11): 2250-2260. PMCID: PMC9826214
44. Barradas M, Fonseca P, Almeida J, Oliveira M, Goncalves H, Primo J, Tavares A, Martins D, Fontes-Carvalho R. Catheter ablation for atypical atrial flutter: characterization and recurrence predictors. Eur Heart J. Oxford Academic; 2023 Nov. 9; 44 (Supplement_2): ehad655.378.
45. Trayanova N A, Popescu D M, Shade J K. Machine Learning in Arrhythmia and Electrophysiology. Circ Res. 2021 Feb. 19; 128 (4): 544-566. PMCID: PMC7899082
46. Feeny A K, Chung M K, Madabhushi A, Attia Z I, Cikes M, Firouznia M, Friedman P A, Kalscheur M M, Kapa S, Narayan S M, Noseworthy P A, Passman R S, Perez M V, Peters N S, Piccini J P, Tarakji K G, Thomas S A, Trayanova N A, Turakhia M P, Wang P J. Artificial Intelligence and Machine Learning in Arrhythmias and Cardiac Electrophysiology. Circ Arrhythm Electrophysiol. 2020 August; 13 (8): e007952. PMCID: PMC7808396
47. Zhou J, Cui G, Hu S, Zhang Z, Yang C, Liu Z, Wang L, Li C, Sun M. Graph neural networks: A review of methods and applications. A I Open. 2020 Jan. 1; 1:57-81.
48. Williams S E, Tobon-Gomez C, Zuluaga M A, Chubb H, Butakoff C, Karim R, Ahmed E, Camara O, Rhode K S. Standardized unfold mapping: a technique to permit left atrial regional data display and analysis. J Interv Card Electrophysiol. 2017 October; 50 (1): 125-131. PMCID: PMC5633640
49. Fahmy A S, Neisius U, Chan R H, Rowin E J, Manning W J, Maron M S, Nezafat R. Three-dimensional Deep Convolutional Neural Networks for Automated Myocardial Scar Quantification in Hypertrophic Cardiomyopathy: A Multicenter Multivendor Study. Radiology. 2020 January; 294 (1): 52-60. PMCID: PMC6939743
50. Acosta J, Penela D, Andreu D, Cabrera M, Carlosena A, Vassanelli F, Alarcón F, Soto-Iglesias D, Korshunov V, Borras R, Linhart M, Martínez M, Fernández-Armenta J, Mont L, Berruezo A. Multielectrode vs. point-by-point mapping for ventricular tachycardia substrate ablation: a randomized study. Europace. 2018 Mar. 1; 20 (3): 512-519. PMID: 28069835
51. Stevenson W G, Friedman P L, Sager P T, Saxon L A, Kocovic D, Harada T, Wiener I, Khan H. Exploring postinfarction reentrant ventricular tachycardia with entrainment mapping. J Am Coll Cardiol. 1997 May; 29 (6): 1180-1189. PMID: 9137211
52. Ciaccio E J, Lee T. Isochronal difference mapping: an approach for mapping dynamic changes during reentrant ventricular tachycardia. Pacing Clin Electrophysiol. 2002 December; 25 (12): 1737-1746. PMID: 12520675
53. Konings K T, Kirchhof C J, Smeets J R, Wellens H J, Penn O C, Allessie M A. High-density mapping of electrically induced atrial fibrillation in humans. Circulation. 1994 April; 89 (4): 1665-1680. PMID: 8149534
54. Anter E, Duytschaever M, Shen C, Strisciuglio T, Leshem E, Contreras-Valdes F M, Waks J W, Zimetbaum P J, Kumar K, Spector P S, Lee A, Gerstenfeld E P, Nakar E, Bar-Tal M, Buxton A E. Activation Mapping With Integration of Vector and Velocity Information Improves the Ability to Identify the Mechanism and Location of Complex Scar-Related Atrial Tachycardias. Circ Arrhythm Electrophysiol. 2018 August; 11 (8): e006536. PMID: 30354312
55. Dittrich S, Scheurlen C, van den Bruck J H, Filipovic K, Wörmann J, Erlhöfer S, Schipper J H, Lüker J, Steven D, Sultan A. The omnipolar mapping technology-a new mapping tool to overcome “bipolar blindness” resulting in true high-density maps. J Interv Card Electrophysiol. 2024 Mar. 1; 67 (2): 399-408.
56. Deno D C, Balachandran R, Morgan D, Ahmad F, Masse S, Nanthakumar K. Orientation-Independent Catheter-Based Characterization of Myocardial Activation. IEEE Trans Biomed Eng. 2017 May; 64 (5): 1067-1077. PMID: 27411215
57. Magtibay K, Porta-Sánchez A, Haldar S K, Deno D C, Massé S, Nanthakumar K. Reinserting Physiology into Cardiac Mapping Using Omnipolar Electrograms. Card Electrophysiol Clin. 2019 September; 11 (3): 525-536. PMID: 31400876
58. Hu J, Shen L, Sun G. Squeeze-and-Excitation Networks. 2018 IEEE/CVF Conference on Computer Vision and Pattern Recognition. IEEE; 2018. p. 7132-7141.
59. Wang Y, Huang H, Rudin C, Shaposhnik Y. Understanding How Dimension Reduction Tools Work: An Empirical Approach to Deciphering t-SNE, UMAP, TriMap, and PaCMAP for Data Visualization. J Mach Learn Res. 2021; 22 (201): 1-73.
60. Di Biase L, Burkhardt J D, Lakkireddy D, Carbucicchio C, Mohanty S, Mohanty P, Trivedi C, Santangeli P, Bai R, Forleo G, Horton R, Bailey S, Sanchez J, Al-Ahmad A, Hranitzky P, Gallinghouse G J, Pelargonio G, Hongo R H, Beheiry S, Hao S C, Reddy M, Rossillo A, Themistoclakis S, Dello Russo A, Casella M, Tondo C, Natale A. Ablation of Stable VTs Versus Substrate Ablation in Ischemic Cardiomyopathy: The VISTA Randomized Multicenter Trial. J Am Coll Cardiol. 2015 Dec. 29; 66 (25): 2872-2882. PMID: 26718674
61. Cantwell C D, Mohamied Y, Tzortzis K N, Garasto S, Houston C, Chowdhury R A, Ng F S, Bharath A A, Peters N S. Rethinking multiscale cardiac electrophysiology with machine learning and predictive modelling. Comput Biol Med. 2019 January; 104:339-351. PMCID: PMC6334203
62. Missel R, Gyawali P K, Murkute J V, Li Z, Zhou S, AbdelWahab A, Davis J, Warren J, Sapp J L, Wang L. A hybrid machine learning approach to localizing the origin of ventricular tachycardia using 12-lead electrocardiograms. Comput Biol Med. 2020 November; 126:104013. PMCID: PMC7606703
63. Leiner T, Rueckert D, Suinesiaputra A, Baeßler B, Nezafat R, Išgum I, Young A A. Machine learning in cardiovascular magnetic resonance: basic concepts and applications. J Cardiovasc Magn Reson. 2019 Oct. 7; 21 (1): 61. PMCID: PMC6778980
64. Chen C, Qin C, Qiu H, Tarroni G, Duan J, Bai W, Rueckert D. Deep Learning for Cardiac Image Segmentation: A Review. Front Cardiovasc Med. 2020 Mar. 5; 7:25. PMCID: PMC7066212
65. Zhang S, Tong H, Xu J, Maciejewski R. Graph convolutional networks: a comprehensive review. Comput Soc Netw. 2019 Nov. 10; 6 (1): 11. PMCID: PMC10615927
66. Kipf T N, Welling M. Semi-Supervised Classification with Graph Convolutional Networks [Internet]. arXiv [cs.LG]. 2016. Available from: http.//arxiv.org/abs/1609.02907
67. Qi C R, Su H, Mo K, Guibas L J. PointNet: Deep Learning on Point Sets for 3D Classification and Segmentation Available from: [Internet]. arXiv [cs.CV]. 2016. http://arxiv.org/abs/1612.00593
68. Qi C R, Yi L, Su H, Guibas L J. PointNet++: Deep Hierarchical Feature Learning on Point Sets in a Metric Space from: [Internet]. arXiv [cs.CV]. 2017. Available http://arxiv org/abs/1706.02413
69. Khater I M, Aroca-Ouellette S T, Meng F, Nabi I R, Hamarneh G. Caveolae and scaffold detection from single molecule localization microscopy data using deep learning. PLOS One. 2019 Aug. 26; 14 (8): e0211659. PMCID: PMC6709882
70. Yang J, Li Z, Zhan P, Li X, Wang K, Han J, Yang P. Proximal femur parameter measurement via improved PointNet+. Int J Med Robot. 2023 June; 19 (3): e2494. PMID: 36527276
71. Friedman D J, Overmann J A, Fish J M, Gaeta S A, Tranter J H, Thao R, Piccini J P. Continuous and discontinuous radiofrequency energy delivery on the atrial free wall: Lesion transmurality, width, and biophysical characteristics. Heart Rhythm O2. 2021 December; 2 (6Part A): 635-641. PMCID: PMC8703143

What is claimed is:

1. A method for providing or facilitating an electrogram feature set associated with an object, comprising:
receiving image information for the object; and
determining at least one characteristic of at least one portion of the object based on the image information and a time series voltage trace.

2. The method of claim 1, wherein the determination of the at least one characteristic uses one or more distances between sampling points between adjacent sections of the object.

3. The method of claim 1, wherein the determination of the at least one characteristic is performed by a machine learning procedure.

4. The method of claim 1, wherein the at least one characteristic is related to a reentry isthmus location.

5. The method of claim 1, wherein the at least one characteristic is determined from an electrogram of a patient undergoing a catheter ablation procedure of an atrial tachycardia.

6. The method of claim 1, wherein the at least one characteristic is determined from an electrogram of a patient undergoing a catheter ablation procedure of a ventricular tachycardia.

7. A non-transitory computer accessible medium which includes software thereon for providing or facilitating an electrogram feature set associated with an object, wherein, when at least one computer processor execute the software, the computer processor is configured to perform the procedures, comprising:
receiving image information for the object; and
determining at least one characteristic of at least one portion of the object based on the image information and a time series voltage trace.

8. The non-transitory computer accessible medium of claim 7, wherein the determination of the at least one characteristic uses one or more distances between sampling points between adjacent sections of the object.

9. The non-transitory computer accessible medium of claim 7, wherein the determination of the at least one characteristic is performed by a machine learning procedure.

10. The non-transitory computer accessible medium of claim 7, wherein the at least one characteristic is related to a reentry isthmus location.

11. The non-transitory computer accessible medium of claim 7, wherein the at least one characteristic is determined from an electrogram of a patient undergoing a catheter ablation procedure of an atrial tachycardia.

12. The non-transitory computer accessible medium of claim 7, wherein the at least one characteristic is determined from an electrogram of a patient undergoing a catheter ablation procedure of a ventricular tachycardia.

13. A system for providing or facilitating an electrogram feature set associated with an object, comprising:
at least one computer processor which is configured to:
receive image information for the object; and
determining at least one characteristic of at least one portion of the object based on the image information and a time series voltage trace.

14. The system of claim 13, wherein the determination of the at least one characteristic uses one or more distances between sampling points between adjacent sections of the object.

15. The system of claim 13, wherein the determination of the at least one characteristic is performed by a machine learning procedure.

16. The system of claim 13, wherein the at least one characteristic is related to a reentry isthmus location.

17. The system of claim 13, wherein the at least one characteristic is determined from an electrogram of a patient undergoing a catheter ablation procedure of an atrial tachycardia.

18. The system of claim 13, wherein the at least one characteristic is determined from an electrogram of a patient undergoing a catheter ablation procedure of a ventricular tachycardia.

19. A method for localizing a reentry isthmus, comprising:
receiving image information for an object; and
generating a probability map for the object based on a plurality of characteristics related to an electrogram feature set, and using the image information.

20. The method of claim 19, wherein the probability map is generated using a graph-convolutional neural network (GCN).

21. The method of claim 20, wherein the GCN generates the probability map at least partially based on a plurality of irregularly spaced electrogram sampling points.

22. The method of claim 20, wherein the GCN is trained on the plurality of characteristics.

23. The method of claim 19, wherein the probability map is used to predict or generate a location and a shape of the reentry isthmus.

24. A non-transitory computer accessible medium which includes software thereon for localizing a reentry isthmus, wherein, when at least one computer processor execute the software, the computer processor is configured to perform the procedures, comprising:
receiving image information for an object; and
generating a probability map for the object based on a plurality of characteristics related to an electrogram feature set, and using the image information

25. The non-transitory computer accessible medium of claim 24, wherein the probability map is generated using a graph-convolutional neural network (GCN).

26. The non-transitory computer accessible medium of claim 25, wherein the GCN generates the probability map at least partially based on a plurality of irregularly spaced electrogram sampling points.

27. The non-transitory computer accessible medium of claim 25, wherein the GCN is trained on the plurality of characteristics.

28. The non-transitory computer accessible medium of claim 24, wherein the probability map is used to predict or generate a location and a shape of the reentry isthmus.

29. A system for localizing a reentry isthmus, comprising:
at least one computer processor which is configured to:
receive image information for an object; and
generate a probability map for the object based on a plurality of characteristics related to an electrogram feature set, and using the image information.

30. The system of claim 29, wherein the probability map is generated using a graph-convolutional neural network (GCN).

31. The system of claim 30, wherein the GCN generates the probability map at least partially based on a plurality of irregularly spaced electrogram sampling points.

32. The system of claim 30, wherein the GCN is trained on the plurality of characteristics.

33. The system of claim 29, wherein the probability map is used to predict or generate a location and a shape of the reentry isthmus.
